SEQUENCE LISTING (7 sequences)

SEQ ID NO: 1 - DNA, 21 bp, artificial
  primer
ctaaagcatgtttcttggcta

SEQ ID NO: 2 - DNA, 20 bp, artificial
  primer
agaagcagttcctgattcca

SEQ ID NO: 3 - DNA, 20 bp, artificial
  primer
aaagaggctcacataactca

SEQ ID NO: 4 - DNA, 20 bp, artificial
  primer
aaataaggtccagcccaatg

SEQ ID NO: 5 - DNA, 9 bp, mouse
  misc_feature (4)..(6): n is a, c, g, or t
ttcnnngaa

SEQ ID NO: 6 - DNA, 9 bp, mouse
  misc_feature (4)..(6): n is a, c, g, or t
aagnnnctt

SEQ ID NO: 7 - DNA, 9 bp, artificial
  primer
ttccgggaa

(19) United States
(12) Patent Application Publication
(10) Pub. No.: US 2010/0178652 A1
(43) Pub. Date: Jul. 15, 2010

(54) Materials and Methods for the Detection, Prevention and Treatment of Autoimmune Disease

(76) Inventors: Sally A. Litherland, Satellite Beach, FL (US); Marcia McDuffie, Charlottesville, VA (US); Laurence Morel, Newberry, FL (US); Federica Seydel, Livermore, CA (US); Erin Garrigan, Gainesville, FL (US); Nicole S. Belkin, Gainesville, FL (US); Bryan Stutevoss, Lake City, FL (US); Michael J. Clare-Salzler, Gainesville, FL (US); Mark A. Atkinson, Gainesville, FL (US); Clive Henry Wasserfall, Gainesville, FL (US); Ammon B. Peck, Gainesville, FL (US); Abdolreza Davoodi-Semiromi, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/522,155

(22) PCT Filed: Jan. 7, 2008
(86) PCT No.: PCT/US08/00248

§ 371 Date: Jan. 25, 2010

Related U.S. Application Data
(60) Provisional application No. 60/878,958, filed on Jan. 5, 2007.

(51) Int. Cl. C12Q 1/68

(52) U.S. Cl. 435/6

(57) ABSTRACT

The subject invention provides materials and methods for the detection, prevention, and treatment of diabetes and other autoimmune conditions.

### CROSS-REFERENCE TO RELATED APPLICATIONS

This application claims the benefit of U.S. Provisional Application Ser. No. 60/878,958, filed on Jan. 5, 2007, which is incorporated herein by reference in its entirety.

### GOVERNMENT SUPPORT

The subject matter of this application has been supported in part by U.S. Government Support under NIH/NIDDK under Grant Nos. KO1 DK002947 and R21DK02082 AND under NIH/NIAID under Grant No. PO1 AI22881. Accordingly, the U.S. Government has certain rights in this invention.

### BACKGROUND OF THE INVENTION

Autoimmunity is the failure of an organism to recognize its own constituent parts as “self”, which results in a pathological immune response against the organism's own cells and tissues. Diseases that result from such an aberrant immune response are termed “autoimmune diseases”. Prominent examples of autoimmune diseases include diabetes mellitus type 1, systemic lupus erythematosus (SLE), Siögren's syndrome, Hashimoto's thyroiditis, Graves' disease, and rheumatoid arthritis (RA).

Autoimmune diseases result from a mix of genetic and environment factors which trigger aberrant immune responses and the loss of self-tolerance. Chronic inflammation and loss of antigen presenting cell (APC) function are two such factors underlying many, if not all, autoimmune diseases.

Diabetes mellitus is a family of disorders characterized by chronic hyperglycemia and the development of long-term vascular complications. This family of disorders includes type 1 diabetes, type 2 diabetes, gestational diabetes, and other types of diabetes.

Patients with type 1 diabetes are unusually prone to other diseases that have become recognized as having autoimmune origins. These diseases include thyroiditis or Hashimoto's disease, Graves' disease, Addison's disease, atrophic gastritis and pernicious anemia, celiac disease, and vitiligo (Maclaren, N. K. [1985] Diabetes Care 8(suppl.):34-38).

Type I diabetes is characterized by an initial leukocyte infiltration into the pancreas that eventually leads to inflammatory lesions within islets, a process called “insulitis”. Type 1 diabetes is distinct from non-insulin dependent diabetes (NIDDM) in that only the type 1 form involves specific destruction of the insulin producing beta cells of the islets of Langerhans. The destruction of beta cells appears to be a result of specific autoimmune attack, in which the patient's own immune system destroys the beta cells, but not the surrounding alpha cells (glucagon producing) or delta cells (somatostatin producing) that comprise the pancreatic islet. The progressive loss of pancreatic beta cells results in insufficient insulin production and, thus, impaired glucose metabolism with attendant complications.

As with other autoimmune diseases, autoimmune diabetes is caused by a combination of genetic and environmental factors, most of which are unknown. Each individual has unique combination of these factors that affects their susceptibility to disease. Understanding how environmental triggers activate diabetes susceptibility genes (Idd loci) is crucial to uncovering how to control or prevent the disease. One set of genetic factors involved in type 1 diabetes are genes regulating immune cell functions.

It has previously been demonstrated in several autoimmune diseases, including IDD, systemic lupus erythematosus (SLE), rheumatoid arthritis (RA), multiple sclerosis (MS), and autoimmune thyroid disease, that antigen-presenting cells (APCs) such as monocytes and macrophages are dysfunctional in their ability to activate T lymphocytes (Via, C. S. et al. [1993] J. Immunol. 151:3914-3922; Serreze, D. [1993] FASEB J. 7:1092-1096; Rasanen, L. et al. [1988] Clin. Exp. Immunol. 71:470-474; Hafler, D. A., et al. [1985] J. Neuroimmunol. 9:339-347).

Myeloid cell differentiation gives rise to 3 populations of professional APC in the immune system: monocytes, macrophages, and dendritic cells (DC). Sequential signaling from IL-3, granulocyte-macrophage-, macrophage-, and granulocyte-colony stimulating factors (GM-, M-, and G-CSF, respectively) sets the microenvironment necessary for bone marrow myeloid precursor cells to differentiate and function [1]. Tight control of the timing and level of each cytokine in myeloid cell microenvironment is needed in order for myeloid cell differentiation to mature cells, and to prevent the pre-mature activation of genes involved in macrophage functional activation, such as COX 2/PGS2, which can promote inflammation.

GM-CSF has a unique dual influence in myeloid cells. First, GM-CSF acts as a differentiation stimulus in myeloid hematopoiesis, and then later it acts as an activation stimulus in mature monocytes and macrophages. Both overexpression and knock-out deletion of GM-CSF in mice can lead to dysregulation of myeloid differentiation and autoimmune disease [2]. This switch in GM-CSF function relies heavily on a change in responsiveness of cells to GM-CSF before and after M-CSF stimulation [3], and on the ability of GM-CSF to activate different STAT5 isoforms at different stages of myeloid cell maturation[4,5].

Work by Piazza et al (2000) [4] suggests that STAT5 isoform changes can act as key cytokine and growth factor-inducible regulatory ‘switches’ for myeloid differentiation and activation. In early myeloid differentiation stages, IL-3 and GM-CSF can induce signaling through both full-length STAT5A (94k-96k) and B (94-92k) isoforms, which act as adaptor molecules for histone acetylases and deacetylases, as well as through truncated isoforms (77k and 80k) that lack the transcriptional activator motif containing a binding site for acetylase enzymes [4-8]. These truncated STAT5 isoforms are not derived from splice variations as seen in other STAT proteins, but produced post-translationally by the actions of a myeloid=specific nuclear serine protease [9,10].

As myeloid cells mature to macrophages and granulocytes, they down regulate the protease and lose their ability to produce truncated STAT5 isoforms, so that signaling through M-CSF and G-CSF signals in matured and activated cells act only through the full-length STAT5 isoforms. [4,11,12]. Thus, during cytokine-induced differentiation, truncated STAT5 isoforms can act as repressors of gene transcription in immature/unactivated cells, while full-length STAT5 isoforms induced in mature/activated cells act as gene transcription activators [4,7,8].

Transfection with genetically engineered dominant negative STAT5 isoform construct blocks myeloid cell precursors at immature stage in differentiation, even if active full-length STAT5 isoforms are present [4]. In contrast, without the dominant negative STAT5 isoforms (i.e., in cells where the protease activity was absent or repressed), GM-CSF and other maturation stimuli can push myeloid cell lines through to maturation.

Serreze et al (1993) [13] found that myeloid APC differentiation is impaired in NOD mouse bone marrow due to a lack of responsiveness to M-CSF. This non-responsiveness was not linked to any defect in M-CSF expression or defect in its receptor, but to unknown problems with M-CSF intracellular signaling. Morin et al (2003) [14] also noted that high GM-CSF can skew NOD myeloid differentiation away from macrophage and DC development, and towards an excess of granulocyte production. Several laboratories have reported disproportion of immature DC compared to mature DC in the diabetic NOD [14-16]. These APC dysfunctional phenotypes suggest that NOD myeloid differentiation and activation are defective at a point or points were the determination of specific cell lineage decisions are made based on the cytokine microenvironment [1,4].

Similar defective myeloid cell phenotypes have been seen in human autoimmune diseases such as type 1 diabetes, SLE, and autoimmune thyroid diseases [13-20]. The result of these defective myeloid cell properties is a loss of professional APC that can efficiently promote tolerance, and regulate appropriate macrophage inflammatory responses [15,21,22]. Loss of these functions sets up an immune microenvironment conducive to immunopathogenesis.

Although knowledge of the immune system has become much more extensive, in recent years, the precise etiology of type 1 diabetes remains a mystery. Furthermore, despite the enormously deleterious health and economic consequences, and the extensive research effort, there currently is no effective means for controlling the formation of this disease.

Type 1 diabetes is currently managed by the administration of exogenous human recombinant insulin. Although insulin administration is effective in achieving some level of euglycemia in most patients, it does not prevent the long-term complications of the disease including ketosis and damage to small blood vessels, which may affect eyesight, kidney function, blood pressure and can cause circulatory system complications.

Because IDD takes several years to develop in an individual, autoimmunity may be firmly established at the time that individuals develop ICAs. Detection of background cellular or genetic factors necessary for the development of autoimmune disease and expressed early in the disease process is of great clinical importance. Detection of these factors would preferably identify individuals before autoimmunity is initiated. Earlier detection would be of great clinical importance in identifying individuals at high risk for disease where the administration of preventative therapies that attempt to preserve the residual insulin-secreting cells are employed.

### BRIEF SUMMARY

The subject invention provides materials and methods for the detection, prevention, and/or treatment of diabetes and other autoimmune conditions. In specific embodiments, the subject invention provides diagnostic procedures that, advantageously, can be used to detect a predisposition to autoimmune disease prior to the time when such disease can be detected using current serological methods and/or with greater accuracy than can be achieved with current methods of detection.

Furthermore, by identifying early critical events in the pathogenesis of autoimmune disease, the current invention facilitates the development of therapeutic compositions and strategies that can intervene in the pathological process thereby reducing the severity, or even preventing, the disease.

In accordance with one embodiment of the subject invention, it has been determined that control of gene expression for myeloid immune cells in NOD mice and diabetic humans' is defective at the level of chromosome structure (chromatin). This ‘epigenetic’ gene regulation can be caused by enzymatic changes in proteins surrounding DNA in chromatin. Specifically, it has been found that GM-CSF, a cytokine involved in myeloid differentiation and function, is found in the Idd 4.3 region and can induce epigenetic changes in gene regulation by activating STAT5 proteins that recruit the enzymes involved in epigenetic protein modification.

In one aspect, the subject invention pertains to use the biomarkers for the detection, prevention, and/or treatment of autoimmune diseases such as diabetes, lupus, and autoimmune thyroid disease. In specific embodiments, the biomarker can be, for example, the STAT5 binding site sequence in regulatory regions of the Csf2 and Ptgs2 genes, STAT5 phosphorylation state, STAT5 binding at these sites, and GM-CSF and COX2/PGS2 production and activities by autoimmune myeloid cells.

The subject invention provides methods for early detection of individuals at-risk for developing type 1 diabetes, or other autoimmune conditions. In one embodiment, the diagnostic methods of the subject invention involves the evaluation of three myeloid antigen presenting cells (APC) phenotypes. These three phenotypes are STAT5 dysfunction, GM-CSF overproduction, and aberrant PGS/COX2 expression/activity. In one embodiment the diagnosis involves only an evaluation of these phenotypes. In other embodiments the evaluation involves the associated genotypes in addition to, or instead of, the phenotypes. In one embodiment, the analyses are performed concurrently via a flow cytometric-based assay. Advantageously, this can be done with even small volume samples.

A further aspect of the subject invention pertains to the use of, for example, nutritional intervention to delay or prevent the onset of clinical symptoms of Type 1 diabetes for individuals that are determined, based on the methods of the subject invention, to be at risk for developing type 1 diabetes.

### BRIEF DESCRIPTION OF THE DRAWINGS

FIG. 1 Analysis of GM-CSF-Induced STAT5 Binding and PGS2/COX2 Activity in Mouse Myeloid cells. A. Chromosome 11 and 1 maps showing the regions of genomic DNA of NOD (white) origin or of non-autoimmune control strains, C57BL/6 (black) and C57L (grey) congenically bred onto B6.NOD mouse strains, which have C57BL/6 genetic backgrounds and NOD.L mice strains, which have NOD genetic backgrounds. Phenotype summary is listed below the maps and approximate map locations for regions of interest are indicated to the left of the maps (not drawn to scale). STAT5 in situ 1 chromatin binding studies were done with 5 million bone marrow cells from each strain were cultured with or without 1000 U/ml GM-CSF for 2-24 hr at 378 C/5% CO2. B.) When primers amplifying known epigenetic modification sites within the Csf2 promoter were used in bone marrow (BM) myeloid cell anti-STAT5Ptyr-ChlP analyses, we found that at 24 hr, GM-CSF enhanced STAT5 binding to several of these sites in control BM cells of NOD congenic mice carrying NOD DNA in this region. C.) Cultures +/−100 μM Na vanadate for 2 hr at 378 C/5% CO2 were used in anti-STAT5Ptyr/anti-Histone H3 mediated double ChlP protein isolations for western blot analysis of STAT5 associated with histone/chromatin complexes, probed initially with anti-STAT5Ptyr antibodies (upper most panel), then stripped and re-probed with anti-CBP/P300 acetylase (P300), RNA polymerase II (POLII), and acetyl ated histone H3(acetyl H3) antibodies. Bensitometric and Rf value analyses were used to give the approximate size and location of the STAT5 protein complexes and monomers (approximate molecular weights indicated on the left of the figure). The estimated molecular size of bands indicate that they may represent formaldehyde-crosslinked STAT5 dimer complexes contain all isoforms of STAT5 (STAT5A (A 96K), STAT5B (B 92K), and truncated (T 80K, 77K)), both in homodimers (A-A 192K, B-B 184K, T-T) and heterodimers (A-B, A-T, B-T, T-T) complexes. Only monmeric STAT5A size bands (96K) are detected in the GM-CSF-induced NOD cell cultures at this time point. The position of the precipitating antibodies heavy chain (50K) and light chain (25K) was determined from the extract minus sham control (sh) and size standards (indicated but not shown). Treatment/lane key: 0=untreated, V=vanadate alone, G=GM-CSF alone, VG=vanadate & GM-CSF supplemented 2 hr cultures. *show lanes where STAT5Ptyr, P300, POLII, and acetyl Histone H3 were detected together. D.) Real Time PCR analysis of DNA containing the Ptgs2 gene enhancer in anti-STAT5Ptyr-ChlP NOD, C56BL/6, NOD.LC11B). E.) ELISA of PGE2 production shows that congenic corretion of Csf2 promoter affects PGS2/COX2 activity that is reconstituted with GM-CSF activates both its own expression and that of PGS2/COX2 through effects on STAT5 binding sites (bold & boxed) and half-sites (in yellow)[14]. G.) Histological images of pancreatic tissue from NOD congenics shows insulitis in only 2 strains, the NOD. LC11E and the bicongenic B6.NODC11bxC1tb, both of which have their Csf2 promoter & Ptgs2 region from NOD though their Csf2 genes are from the C57BL/6 non-autoimmune strain.

FIG. 2 GM-CSF/M-CSF-cytokine-induced myeloid differentiation in vitro.

FIG. 3 shows flow cytometric analysis of STAT5 phosphorylation in in vitro myeloid differentiation

FIG. 4 shows GM-CSF/M-CSF cytokine switch in vitro myeloid differentiation experiments.

FIG. 5 shows DNA affinity precipitation and immunoprecipitation of STAT5 in GM-CSF-differentiated bone marrow myeloid cells.

FIG. 6 shows chromatin immunoprecipitation (ChIP) analysis of STAT5 binding within the promoter region upstream of the Csf2 gene which encodes for GM-CSF.

FIG. 7 shows a mechanistic model for GM-CSF/M-CSF dysregulation of STAT5 function in epigenetic regulation of myeloid cell gene expression.

FIG. 8 shows myeloid differentiation.

FIG. 9 shows a sequence comparisons for Csf2 promoter region reveals polymorphism in NOD. 9A shows genomic DNA from NOD and C57BL/6 mice were amplified in PCR using primers specific for the −181 to +10 bp region upstream of the Csf2 gene [25]. PCR products run on a 5% agarose gel show a small but reproducible size variation between the two mouse strains. Data are representative of 12 runs of the analysis. 9B shows sequence analysis of the −3 to −969 bp region upstream of the Csf2 gene in NOD and C57BL/6 mouse genomic DNA samples revealed differences in STAT5 binding sites (yellow, boxed), many half or imperfect binding sites (yellow), and a microsatellite DNA insertion (blue) in the region that has a length polymorphism between the two strains. Putative STAT5 binding site NOD polymorphisms are indicated by underlined bold, boxed sequences. * indicate sequence homology between the two strains.

FIG. 10 shows GM-CSF production and STAT5 phosphorylation are aberrantly high in NOD mouse myeloid cells. 10A shows four to five million bone marrow cells and adherence-isolated peritoneal macrophages were cultured without supplementation for 24 hr at 37 C/5% CO2. Cell-free culture supernatants were then analyzed by ELISA and/or Luminex for the presence of GMCSF. GM-CSF concentrations were normalized to pg/million plated cells for comparison. The p values listed were obtained from Mann-Whitney U test analysis of the data. Patterned bars indicate the mean GM-CSF production from NOD samples and open bars the mean of C57BL/6 samples. Error bars represent SEM: 10B shows ex vivo myeloid cells from NOD and C57BL/6 mice (peritoneal macrophages, peripheral blood, and bone marrow cells) were collected and fixed within 4 hr of collection and then analyzed for phosphorylated STAT5 by intracellular flow cytometry. The p values listed were obtained from Mann-Whitney U test analysis of the data. Patterned bars indicate the mean % STAT5Ptyr+/CD1 1b+ cells detected in NOD samples and open bars the mean of C57BL/6 samples. Error bars represent SEM.

FIG. 11 shows STAT5 binding to chromatin increases after GM-CSF stimulation in NOD but not C57BL/6 bone marrow cells. Five million bone marrow cells were cultured with or without 100 μM Na vanadate for 30 min at 378 C/5% CO2. Half of the cultures +/−vanadate were then supplement with 1000 U/ml GM-CSF, and all were incubated for an additional 90 min at 378 C/5% CO2. The cells were then fixed in situ, extracted and sonicated. The sample was split in to 5−1×10⁶ cell aliquots for use in anti-STAT5Ptyr/anti-Histone H3 mediated double ChIP protein isolations for western blot analysis of STAT5 associated with histone/chromatin complexes. Protein isolated from the precipitated chromatin complexes and analyzed by western blot probed initially with anti-STAT5Ptyr antibodies (upper most panel), and then stripped and re-probed with antibodies to detect CBP/P300 acetylase (P300), RNA polymerase II (POLII), and acetyl ated histone H3(acetyl H3). Densitometric and Rf value analyses were used to give the approximate size and location of the STAT5 protein complexes and monomers (approximate molecular weights indicated on the left of the figure). The estimated molecular size of bands indicate that they may represent formaldehyde-crosslinked STAT5 dimer complexes contain all isoforms of STAT5 (STAT5A (A 9 6K), STAT5B (B 92K), and truncated (T 80K, 77K)), both in homodimers (A-A 192K, B-B 184K, T-T) and heterodimers (A-B, A-T, B-T, T-T) complexes. Only monomeric STAT5A size bands (96K) are detected in the GM-CSF-induced NOD cell cultures at this early time point. The position of the precipitating antibodies heavy chain (50K) and light chain (25K) was determined from the extract minus sham control (sh) and size standards (indicated but not shown). Treatment/lane key: 0=untreated, V=vanadate alone, G=GM-CSF alone, VG=vanadate & GM-CSF supplemented 2 hr cultures. Data represents 3 runs of the double ChIP analysis. * show lanes where STAT5Ptyr, P300, POUII, and acetyl Histone H3 were detected.

FIG. 12 shows a macrophage chromatin immunoprecipitation (ChIP) analysis that shows STAT5 binding within the promoter region upstream of the gene which encodes for GM-CSF, Csf2. Four million peritoneal macrophages (PMAC) from NOD and non-autoimmune C57BL/6 mice were incubated for 24 hr without exogenous GM-CSF. Cells were fixed in situ, and extracted for protein-chromatin complexes as described in the Materials & Methods. Extracts divided in 4 parts and immunoprecipitated with anti-STAT5Ptyr antibodies. One hundred nanograms of the ChIP-isolated STAT5Ptyr-associated DNA were analyzed by (12A) conventional PCR/Ethidium bromide agarose gel and by (12B) SybrGreen Real Time PCR for the presence of Csf2 promoter (−181 to +10 bp) DNA sequences. Key: 5P=ChIP anti-STAT5 precipitated DNA, T=total cellular DNA from unprecipitated fixed cell extracts, Ig=ChIP non-specific mouse IgG precipitated DNA, W=DNA-free water control, Data represents results seen in 3 separate runs of the analysis. Patterned bar indicates the mean R value obtained from 3 NOD samples and open bars the mean of 3 C57BL/6 samples. Error bars represent SEM.

FIG. 13 shows ChIP analysis of GM-CSF-induced STAT5 binding upstream at multiple sites within the Csf2 promoter involves DNA secondary structure. Four million cell cultures of NOD and C56BL/6 mouse bone marrow cells (BM) were grown for 24 hr in the presence (GM or G) or absence (0) of 1000 U/ml GM-CSF before being fixed and extracted for ChIP analysis. Aliquots of 100 ng of total DNA extracted from ChIP protein-chromatin complexes precipitated with anti-STAT5 antibodies were amplified using primers to potential epigenetic modification sites within the Csf2 promoter region [24, 25]. 13A. ChIP isolated DNA analyzed in standard PCR (without DMSO or hot start) and run on a 3% agarose ethidium bromide gel. I=ChIP DNA; T=total fixed cell DNA. Schematic of DNA region giving base pair distances from the Csf2 origin of replication (arrow) are not drawn to scale but is included to give the orientation and relative distances between the Csf2 promoter regions amplified on which STAT5 binds and the Csf2 gene coding region. Data representative of 4 separate runs of the assay. 13B. Comparison of NOD and C57BL/6 mouse bone marrow samples of ChIP isolated DNA analyzed by real time PCR using the ‘Region A’ primers (−47 bp to −162 bp region amplified) using hot start and DMSO (with DMSO, white background bars; also depicted in C) to remove secondary structure or standard PCR protocol (without DMSO, grey background bars) as described in the Material & Methods section. Unpatterned bars indicate cells without treatment (0) and hatched bars indicate cells treated with GM-CSF (G). Data representative of 2 (with DMSO) and 4 (without DMSO) sample sets. C. Real time PCR analysis using the hot start and DMSO protocol described in the Materials & Methods to remove secondary structure and the primers depicted in A to amplify and identify Csf2 promoter regions previously identified as epigenetic control sites for Csf2 gene expression. Unpatterned bars indicate cells without treatment (0) and hatched bars indicate cells treated with GM-CSF (G). Data representative of 2 sample sets. D. Real time PCR analysis using the hot start and DMSO protocol described in the Materials and Methods to remove secondary structure and the primers depicted in A to amplify and identify Csf2 promoter regions previously identified as epigenetic control sites for Csf2 gene expression. Unpatterned bars indicate cells without treatment (0) and hatched bars indicate cells treated with GM-CSF (G). Data representative of 2 (combined A-I) and 3 (Promoter −3 to −969 bp) sample sets. The p values indicate one-way ANOVA analysis (above graphs) or Mann-Whitney U tests (on graph) of pairwise comparisons.

### DETAILED DISCLOSURE

The subject invention provides materials and methods for the detection, prevention, and treatment of diabetes and other autoimmune conditions. Specifically exemplified herein are diagnostic procedures that can be used for early detection of a predisposition to autoimmune disease. Furthermore, by identifying early critical events in the pathogenesis of autoimmune disease, the current invention facilitates the development of therapeutic compositions and strategies that can intervene in the pathological process thereby reducing the severity, or even preventing, the disease.

Given the difficulty in manipulating multiple genes by gene therapy, the diversity of environmental factors that may play into autoimmune susceptibility, and the strong probability that an autoimmune pathology will relapse as long as the underlying immune recognition of self-antigens remains, the most effective intervention strategies for curing autoimmune disease lie in preventing activation of susceptibility genes prior to disease onset. In accordance with the subject invention it has been found that the interaction of GM-CSF, STAT5, and PGS2/COX2 phenotypes in autoimmune myeloid cells represents a manifestation of a new mechanism for autoimmune susceptibility. In this mechanism, epigenetic gene regulation via cytokine-induced STAT-mediated chromatin modifications are a central and shared component of the etiology of multiple autoimmune diseases. Epigenetic dysregulation as an etiological mechanism for environmental factors triggering genetic susceptibility represents a paradigm shift in the approach to treatment and prevention of multifactorial diseases. In accordance with the subject invention, understanding this mechanism suggests preventive measures that can be instilled into the susceptible individual's lifestyle as therapeutic components, such as diet, activity, and preventive medicine therapies.

In accordance with the subject invention, it has been determined that GM-CSF regulates STAT5 binding to DNA in the non-coding sequence upstream of Csf2, the gene encoding for GM-CSF, primarily in regions that are epigenetic chromatin modification sites within the Csf2 promoter. Sequence analysis has revealed multiple polymorphisms in the NOD mouse DNA Csf2 promoter, affecting at least 2 STAT5 binding sites.

Specifically, autoimmune STAT5 dysfunction affects Csf2 and Ptgs2 GM-CSF induced-gene expression, thus affecting the timing of responses to GM-CSF and M-CSF. STAT5 binding at recognition sequences upstream of the promoter region of the Csf2 causes repression of gene expression in normal cells and sites of GM-CSF-induced dysregulation of its own gene. STAT5 dysfunction in NOD mouse myeloid cells appears to be related to unproductive interactions with SMRT and P300; whereas, STAT5 dysfunction in NZM myeloid cells may be related to lack of SMRT function in M-CSF and lack of P300 function in GM-CSF responses. These changes in STAT5-associated chromatin regulation lead to a loss of gene repression in NZM and a gain of gene activation in NOD myeloid cells.

Using anti-tyrosine phosphorylated STAT5 (STAT5Ptyr)-mediated chromatin immunoprecipitation (ChIP) and realtime PCR analysis, it was found that in NOD bone marrow cells, GM-CSF stimulated STAT5 binding at these sites increases along with an increase in STAT5-associated histone acetylation, while GM-CSF decreases binding at the same sites in bone marrow cells of the non-autoimmune mouse strain, C57BL/6. These data suggest that GM-CSF-induced STAT5 DNA binding affects its own Csf2 gene's expression through facilitating epigenetic regulation and, thus, can be an underlying cause of both the overexpression of GM-CSF and the prolonged activation of STAT5 seen in autoimmune monocytes and macrophages.

GM-CSF activates STAT5 binding at its own gene promoter within the Idd4.3 region, as well as at the enhancer of Ptgs2 (mouse chr.1), the gene that encodes PGS2/COX2. Since STAT5 acts as an adaptor protein for either deacetylase or acetylase enzymes mediating epigenetic modification of chromatin, the loss of Csf2 gene regulation in autoimmune cells may be mediated by changes in epigenetic control perpetuated by GM-CSF activating persistent STAT5. Increases in Csf2 gene expression may also induce genes that GM-CSF uses STAT5 to regulate, such as Ptgs2.

By congenic isolation of Idd loci and sequence analysis, it has been determined that the NOD-derived sequence for the GM-CSF (Csf2) coding region is not required for diabetes susceptibility. Furthermore, having a non-coding sequence of NOD origin within the Idd4.3 locus <400 bp upstream of the Csf2 coding region is sufficient to reconstitute NOD phenotypes of GM-CSF production, and STAT5 activation. When these regions are of NOD origin, there is also a loss of GM-CSF-induced truncated STAT5 binding at Csf2 and Ptgs2 upstream regulatory sites in bone marrow cells but a gain of full-length STAT5 binding at the same sites in mature macrophages. Since GM-CSF can activate STAT5 to act as an adaptor for both histone acetylase/deacetylase enzymes, these data indicate that phenotypes could be related through an epigenetic regulatory mechanism normally mediated by STAT5. These findings implicate loss of cytokine-induced suppression of epigenetic modification in non-coding regulatory regions as a mechanism for promoting the aberrant expression of genes or genetic regions (e.g., Idd loci) in autoimmune disease, particularly diabetes.

Thus, in accordance with the subject invention it has been found that myeloid cells of NOD mice have abnormally high granulocyte macrophage colony stimulating factor (GM-CSF) expression and persistent activation of signal transducer/activator of transcription 5 (STAT5) isoforms. Once activated, persistence of STAT5 phosphorylation in NOD mature myeloid cells is not affected by inhibition of GM-CSF signaling with blocking antibodies or with the JAK inhibitor AG490. However, NOD STAT5 phosphorylation can be diminished if GM-CSF signaling is blocked in NOD bone marrow precursor cells. Stimulating C57BL/6 non-autoimmune mouse bone marrow cultures with NOD levels of GM-CSF promotes high STAT5 phosphorylation; however, suppressing GM-CSF in these cells also promotes STAT5 phosphorylation to NOD levels, augmented by subsequent M-CSF activation of STAT5.

The materials and methods of the subject invention take advantage of the identification of a crucial temporal/quantitative interplay of GM-CSF and M-CSF signaling on. STAT5 activation in myeloid cells. The persistence of high GM-CSF expression seen in myeloid cells can block both subsequent M-CSF signaling needed for STAT5 downregulation and progression of myeloid differentiation. Thus, the loss of time-critical downregulation of GM-CSF and GM-CSF-activated STAT5 may be blocking subsequent M-CSF and its role in the progression of myeloid differentiation. This block in development of myeloid antigen presenting cell function can have a direct effect on myeloid antigen presenting cell development and thus, contribute to the loss of self tolerance in immunopathogenesis.

One aspect of the current invention provides analysis protocols for the study of components in human peripheral blood cells including successfully adapting STAT5 phosphorylation analysis to mini blood drop analysis as a screening assay, a small blood volume assay for GM-CSF, a genetic biomarker for the STAT5 binding affected UTR of the CSF2 and PTGS2 genes involved in this dysfunction, and a chromatin immunoprecipitation-based assay for STAT5 binding and functional epigenetic changes regulating GM-CSF and COX2/PGS2 expression. Such screening assays can facilitate the early detection of dysfunction in myeloid cell differentiation and function that may lead to immunopathogenesis.

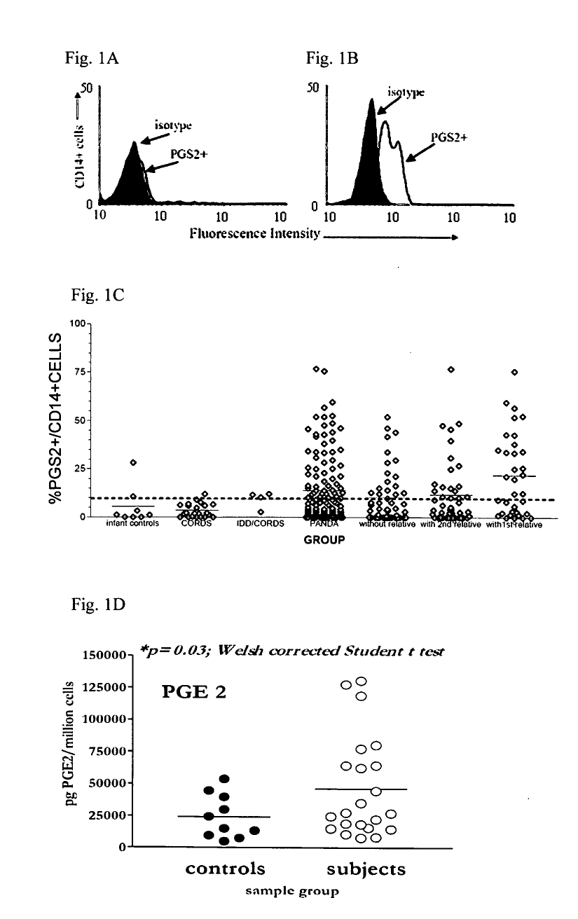

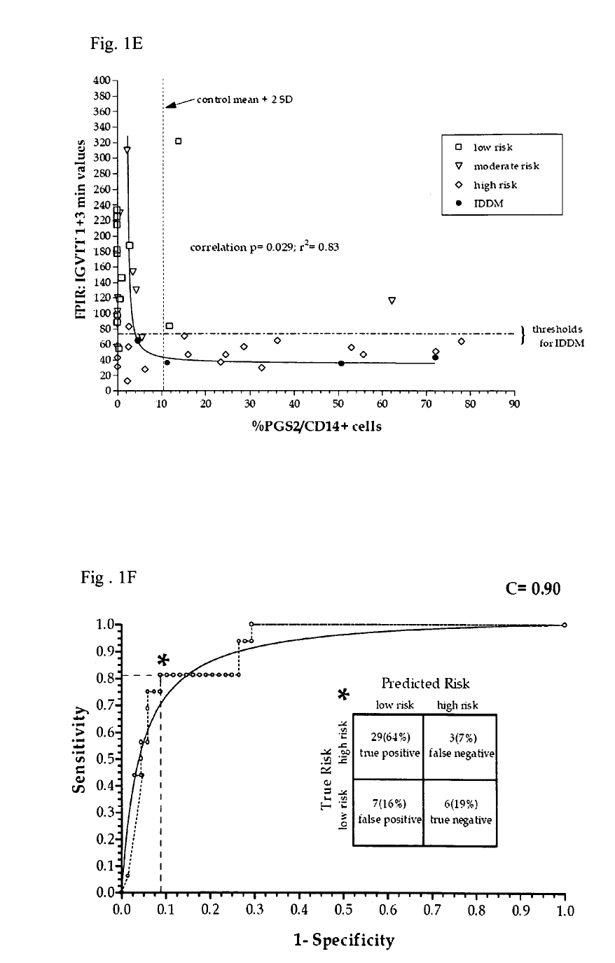

Using established NOD.L and B6.NOD complementary congenic mouse strains and recombinant breeding, the region within ldd4.3 susceptibility loci involved in all three of the autoimmune myeloid cell phenotypes (GM-CSF overexpression, persistent STAT5 activation, and GM-CSF activation of aberrant PGS2/COX2 activity) in the NOD mouse were defined [22,23]. In situ chromatin binding studies in myeloid cells of NOD congenic mice showed an association of a STAT5-mediated epigenetic regulatory mechanism with PGS2/COX2 activation in response to GM-CSF overproduction. These traits appear to be genetically linked through polymorphisms in STAT5 DNA binding sites within the Csf2 promoter (Chr. 11, ldd4.3) and an enhancer upstream of Ptgs2 (Chr. 1, ldd5) (FIG. 1).

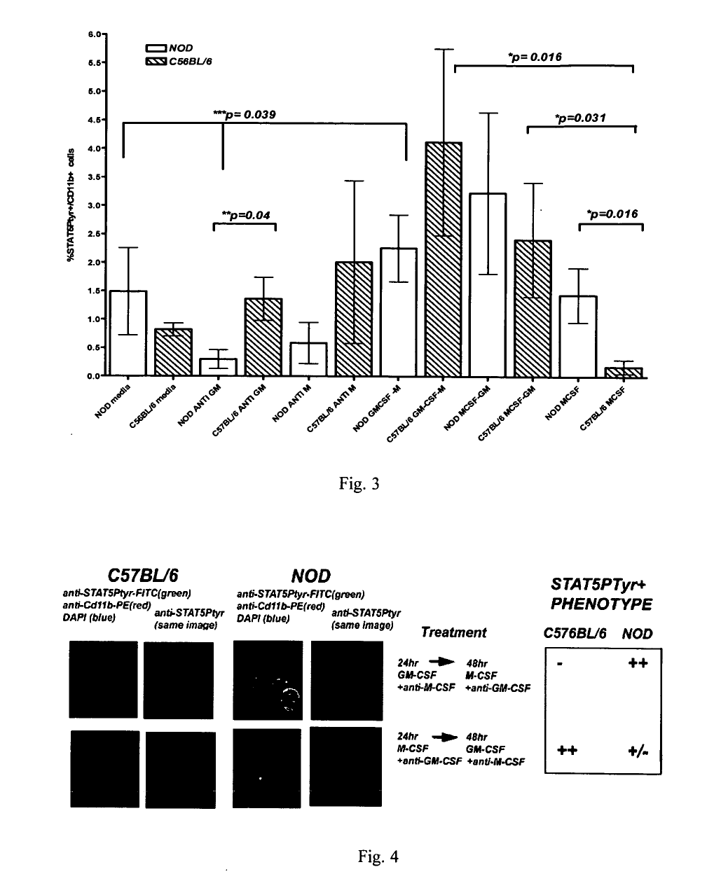

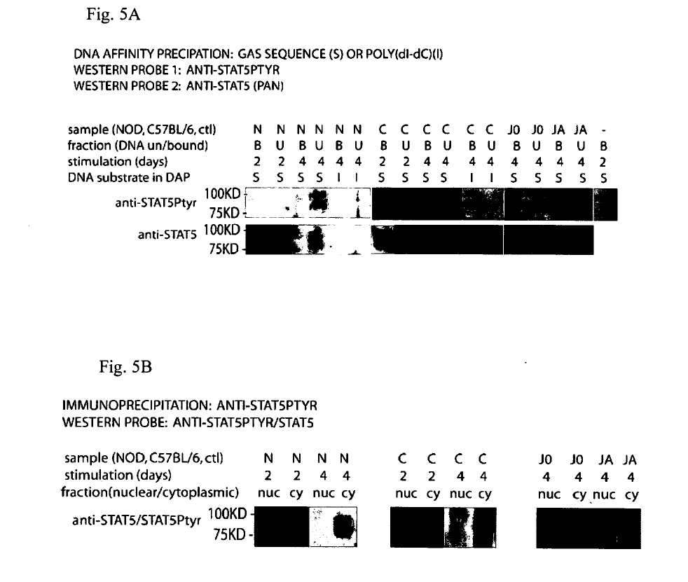

In support of the possibility of Csf2 and Ptgs2 expression responding to GM-CSF through STAT5 effects, STAT5Ptyr binding of these sites, PGS2/COX2 expression and PGE2 production was restored in vitro by GM-CSF in cells from congenic mice which had the Csf2 promoter region corrected (NOD.LC11B but not NOD.LC11E) and also had the Ptgs2 enhancer region of NOD genetic origin (i.e., NOD.LC11B but not B6.NOD.C11B). Bicongenic B6.NODC11bxC1tb mice which have NOD DNA sequences at both Csf2 promoter and Ptgs2 enhancer on an otherwise non-autoimmune C57BL/6 genetic background, also reconstituted these GM-CSF induced phenotypes and developed invasive insultitis (FIG. 1). GM-CSF and PGS2/COX2 overexpression promotes chronic inflammation and poor myeloid APC development through its dysregulation of STAT5 binding at epigenetic regulatory sites needed to control myeloid cell gene expression. These functional defects in NOD cells that echo those seen in at-risk/T1D human monocytes, and are linked not to STAT5 or GM-CSF coding regions, but to conserved epigenetic modification sites in untranscribed regulatory regions controlling gene expression induced by GM-CSF. Sequence analysis of this region indicates that polymorphism(s) in the regulatory upstream chromosomal region of the Csf2 promoter may at least be an essential contributing factor to all 3 phenotypes. The sequence of Csf2 promoter in T1D human DNA has 61% shared homology with NOD mouse, with even more conserved sequence at the GM-CSF-activated STAT5 binding sites within the Csf2 promoter altered in the NOD (FIG. 3F).

Accordingly, the overproduction of GM-CSF by unactivated autoimmune myeloid cells contributes to both aberrant Csf2 and Ptgs2 gene expression through abnormal STAT5 activation and function. These phenotypes found in at-risk subjects and overt T1D patients interact on a gene regulation level to promote the chronic inflammatory milieu needed in immunopathogensis to block tolerance induction and promote immune cell-mediated tissue damage.

In one embodiment of the subject invention, flow cytometric and DNA genotype analyses is designed to detect all three phenotypes for use on minimal blood samples, such as those taken both at birth from PKU blood drop collections or cord blood samples, or from finger prick blood drop samples taken from at-risk/T1D subjects in route blood glucose testing.

Materials and methods for detection, treatment and/or prevention of type 1 diabetes are specifically exemplified herein; however, use in conjunction with other pathological autoimmune conditions is contemplated according to the subject invention. Other autoimmune conditions to which the materials and methods of the subject invention may be applied include, but are not limited to, rheumatoid arthritis, multiple sclerosis, thyroiditis, inflammatory bowel disease, Addison's disease, pancreas transplantation, kidney transplantation, islet transplantation, heart transplantation, lung transplantation, and liver transplantation.

In accordance with the subject invention, autoimmune monocytes and macrophages from human patients and mouse models of autoimmune disease have three potentially linked defects that contribute to their dysfunction in immunopathogenesis: Aberrantly high COX2/PGS2 expression and activity, high GM-CSF autocrine production and hyper-sensitivity to GM-CSF activation, and persistent STAT5 phosphorylation and dysregulation of STAT5 function after GM-CSF activation.

In one embodiment, the diagnostic methods of the subject invention involve the evaluation of three myeloid antigen presenting cells (APC) phenotypes. These three phenotypes are STAT5 dysfunction, GM-CSF overproduction, and aberrant PGS/COX2 expression/activity. In one embodiment the diagnosis involves only an evaluation of these phenotypes, in other embodiments the evaluation involves the associated genotypes in addition to, or instead of, the phenotypes. In one embodiment, the analyses are performed concurrently via a flow cytometric-based assay. Advantageously, this can be done with even small volume samples.

The current assays for PGE2 and GM-CSF analysis in plasma or serum suffer from many sources of potential non-autoimmune related activation (e.g., LPS contamination, cell adherence, NSAID or oral contraceptive treatment, female hormone cycle). These sample handling issues can interfere with interpretation of the assays, thus limiting the flexibility of the assay for accurate measurement. In contrast, STAT5 phosphorylation analysis as described herein does not suffer from the instability or activation limitations of the other biomarkers, and is not adversely affected by female hormone cycle or subject NSAID or oral contraceptive use.

Thus, in one embodiment of the subject invention, the STAT5 phosphorylation analysis is useful either as a stand-alone assay or with a confirmatory analysis for PGS2/COX2 and GM-CSF expression in autoimmune cells. One aspect of the subject invention combines analyses for PGS2/COX2, GM-CSF, and STAT5Ptyr into a small volume flow cytometric analysis using brefeldin or ianomyosin/PMA to block GM-CSF secretion prior to intracellular labeling. By analyzing the three interactive phenotypes simultaneously on the same cells it is possible to amplify, simplify, and increase the biomarker assay's applicability to diagnostic T1D risk screening. Flow cytometric analyses of intact intracellular components (PGS2/COX2, STAT5Ptyr) coupled with detection of intracellular GM-CSF, and analysis of GM-CSF and PGE2 in plasma/serum by Luminex/ELISA can be used to improve the volume required for the tests as well as allow for internal collaborative analyses to confirm that the expression of these parameters are due to the T1D risk potential of the sample and not other possible sources of variation.

A major advantage of combining these analyses is the ability to achieve a more specific assessment of APC dysfunction at multiple but related levels (each assay component acting as a verification test for the others) and its application at routine blood drop collection times such as PKU testing in neonates and glucose/HbA1C testing in clinical patients.

### Methods and Materials

### Mouse Strains & Disease Monitoring:

Eight to twelve week old NOD and C57BL/6 female mice (Jackson Laboratories stock) were used for all studies. These strains were maintained as breeding stock in our Pathology Department SPF colony, in microisolator cages with food and water ad libium. All procedures were conduced according to IACUC approved protocols B083 and D754. A minimum of 3 animals per strain were used per experimental treatment run. Data presented are the results of three (DAP, IP) to five (Flow, fluorescent imaging) separate runs of the experiments.

### Peritoneal Macrophage Collection:

Mice were euthanized according to IACUC approved protocols using over-anesthetization and cervical dislocation. Ice cold RPMI medium supplemented with 10′% fetal calf serum and 1% antibiotic/antimycotic mix (Cellgro, Mediatech) was injected into the peritoneal cavity of the mouse carcass to lavage the macrophage from the abdominal cavity and organs. The lavage fluid was then collected via syringe and held on ice until use. Lavage cells were washed by centrifugation (600×g, 5 min, 10 C) in fresh cold sterile media. Red blood cell (RBC) contamination in samples were lysed by incubation in non-isotonic buffer and the remaining white blood cells plated on tissue culture dishes for adherence purification or used immediately for flow cytometric/ChIP analyses. Plated cells will be held 1 hr at 37 C/5% C0₂ before non-adherent cells are flushed from the wells with successive washes of cold sterile 1×PBS. The remaining adherent cells re-fed with fresh sterile media for up to 48 hr at 37 C/5% C0₂. After incubation, half of media volume from these cultures was collected and frozen at −80 C for later analysis of GM-CSF by Luminex and ELISA. Cells were fixed in situ with 1% (v/v C_{f}) formaldehyde added in the remaining media for 10 min at 37 C, then washed with 1×PBS, and sonicated in SDS Lysis Buffer(Upstate Biotech)+protease & phosphatase inhibitors(Roche) for later analysis in ChIP.

### Bone Marrow Differentiation Culture & Sample Preparation:

Long bones from the legs of mouse carcasses were collected into cold media and cleaned of soft tissue with a sterile scalpel. The ends of each bone were then cut off and the marrow flushed into a sterile tube by cold medium injected into the bone through a 30-gauge needle. The marrow samples were washed with cold media by centrifugation and RBC in samples lysed by incubation in non-isotonic buffer. The remaining bone marrow cells are then plated on tissue culture dishes and fed with fresh sterile media with or without 1000 U/ml of GM-CSF and/or anti-M-CSF blocking antibodies for 24-48 hr then washed and re-fed with 500 U/ml M-CSF and/or anti-GM-CSF blocking antibodies for culture up 2 more days at 37 C/5% C0₂. After incubation, half of media volume from these cultures will be collected and frozen at −80 C for later analysis of GM-CSF and PGE2 by Luminex and ELISA. An aliquot of cells will be taken for phenotypic identification and phosphotyrosine STAT5 analysis by flow cytometry. The rest of the cultured cells were washed with 1×PBS, and extracted for nuclear and cytoplasmic subcellular fractionation as previously described [20].

### Flow Cytometric & Deconvolution Image Analysis:

Flow cytometric analysis of fluorescently-conjugated antibody surface and intracellular binding will be used to phenotypically identify myeloid cells and quantify the level of STAT5 tyrosine phosphorylation in cells ex vivo and after in vitro stimulation. Cells will be first labeled with anti-CD11b and anti-IgM antibody conjugates to identify monocytes and macrophages (CD11b+/IgM−) in the sample, then fixed with 2% (v/v C_{f}) formaldehyde and permeabilized using 0.5% saponin in a high protein, isotonic buffer [17-20,23]. Intracellular staining of STAT5Ptyr will be done using APC-conjugated anti-STAT5Ptyr specific monoclonal antibodies (Upstate Biotech, conjugated using Prozyme APC-labeling kit). This method is well developed in our laboratory and has shown good consistency in current studies [17-20,23]. We will analyze the percentage of positive myeloid cells (% STATPtyr+/CD11b+) as well as mean fluorescence (MF) to assess both the number of cells in the myeloid population with activated STAT5 and the relative amount of phosphorylated STAT5 per cells after treatment. After flow analysis, the labeled cells are centrifuged onto slides and stain them for chromatin (DAPI, Molecular Probes). These cells were used for imaging by deconvolution microscopy as previously described and then used 3 dimensional projection rotational analysis to identify subcellular location of activated STAT5.

### DNA Affinity Precipitation & Immunoprecipitation Western Blot Analysis of STAT5 Phosphorylation and DNA Binding Isoforms

Nuclear and cytoplasmic protein extracts were reacted with FITC-CTP or FITC-UTP-labeled Ptgs2 GAS sequence DNA (Yamaoka, K. T. Otsuka, H. Niiro, Y. Arinobu, Y. Nihho, N. Hamasaki, and K. Izuhara, 1998 “Activation of STAT5 by lipopolysaccharide through granulocyte-macrophage colony-stimulating factor production in human monocytes” J Immunol. 160: 838-845) as previously described (Litherland, S. A., T. X. Xie, K. M. Grebe, A. Davoodi-Semiromi, J. Elf, N. S. Belkin, L. L. Moldawer, and M. J. Clare-Salzler, 2005 “Signal Transduction Activator of Transcription S (STAT5) Proteins are Dysfunctional in Autoimmune Monocytes & Macrophages” J. Autoimmunity. 24: 297-310). DNA-protein complexes were then immunoprecipitated with anti-FITC antibodies (Amersham) coupled to Protein G agarose beads (Upstate Biotech) overnight at 4 C. The precipitated bead complexes were washed twice with 1×PBS (Cellgro, Mediatech) by centrifugation at 600×g for 5 min. Protein in the pelleted complexes were released by boiling in 1× Leammili buffer and analyzed in STAT5 IP/Western blot analysis using anti-phosphotyrosine STAT5 (STAT5Ptyr, Upstate Biotech) or anti-STAT5 (pan, Santa Cruz) antibodies that recognize all four isoforms as the primary probe antibodies as previously described (Litherland, S. A., T. X. Xie, K. M. Grebe, A. Davoodi-Semiromi, J. Elf, N. S. Belkin, L. L. Moldawer, and M. J. Clare-Salzler, 2005 “Signal Transduction Activator of Transcription 5 (STAT5) Proteins are Dysfunctional in Autoimmune Monocytes & Macrophages” J. Autoimmunity. 24: 297-310; and Litherland, S. A., K. M. Grebe, N. S. Belkin, E. Paek, J. Elf, M. Atkinson, L. Morel, M. J. Clare-Salzler, and M. J. McDuffie, 2005 “Nonobese diabetic mouse congenic analysis reveals chromosome 11 locus contributing to diabetes susceptibility, macrophage STAT5 dysfunction, and GM-CSF overproduction” J Immunology. 175(7): 4561-4565).

### Chromatin Immunoprecipitation (ChIP) Analysis of STAT5 Binding at the Csf2 Promoter

Four million cells from bone marrow cultures or ex vivo peritoneal macrophages were fixed in situ with 1% formaldehyde in 1×PBS (methanol-free Sigma-Aldrich) for 10 min at 37 C prior to lysis with High SDS Lysis Buffer (Upstate Biotech): Activated and Unactivated Jurkat cell extracts supplied by the antibody manufacturer (Upstate Biotech) were used for positive controls for the assays. The fixed lysates were sonicated to disrupt membranes and shear chromatin to approximately 1000 bp fragments then frozen until analysis. Once thawed, the samples were divided into 4 aliquots for each run of the analysis: 3 for ChIP, 1 for total cell analysis. The aliquots for IP were pre-cleared with salmon sperm DNA Protein A agarose beads (Upstate Biotech), then incubated overnight at 4 C with anti-STAT5Ptyr antibodies (Upstate Biotech). After incubation, the antibody-bound chromatin complexes were precipitated using salmon sperm DNA Protein A agarose beads, and washed extensively with a series of increasing stringency buffers (low salt, high salt, LiCl, TE; ChIP reagent kit, Upstate Biotech). A non-specific antibody control (mouse IgG, UpState Biotech) and a no extract sham IP were run in parallel as negative assay controls. For each sample, half the total cell aliquot and one ChIP-isolated aliquot were denatured and run on 4-20% SDS PAGE for later western blot analysis with anti-STAT5Ptyr, then re-probed with antibodies to detect protein-protein interactions in the chromatin complex between STAT5 and acetylated histone 3 (Upstate Biotech). The remaining half of the total and ChIP aliquots were dissociated from the beads in SDS-Bicarbonate buffer. NaCl is then added to a final concentration of 500 mM and the formaldehyde crosslinks were reversed by 4 hr incubation at 65 C. DNA was purified from these aliquots for PCR amplification and sequence analysis of Csf2 promoter sequences known to be involved in epigenetic regulation of the Csf2 gene (Ito, K., P. J. Barnes, and I. M. Adcock, 2000 “Glucocortoid Receptor Recruitment of Histone Deacetylase 2 Inhibits Interleukin-1b-Induced Histone H4 Acetylation on Lysines 8 and 12” Mol. Cell. Bio. 20(18): 6891-6903; and Chen, X., J. Wang, D. Woltring, S. Gerondakis, and M. F. Shannon, 2005, “Histone Dynamics on the Interleukin-2 Gene in Response to T-Cell Activation” Mol Cell Biol. 25(8): 3209-3219).

Following are examples which illustrate procedures for practicing the invention. These examples should not be construed as limiting. All percentages are by weight and all solvent mixture proportions are by volume unless otherwise noted.

### EXAMPLE 1

For cytokine switch differentiation experiments; NOD and C57BL/6 mouse bone marrow in vitro differentiation cultures stimulating cells with one cytokine (1000 U/ml GM-CSF or 500 U/ml M-CSF) while blocking the other with blocking antibodies (anti-GM-CSF or anti-M-CSF, 2 μg/ml) for 24 hr, then maintain or reverse the treatment for the next 24 to 48 hr to stimulate with the other cytokine and block the first with antibodies. Bone marrow cells can differentiate to macrophage phenotypes within 4 days culture in M-CSF or 4-7 days in GM-CSF [26]. These experiments were designed to mimic or reverse the natural order of cytokine signaling early in myeloid differentiation at a point prior to mature macrophage, roughly approximating unactivated monocyte development.

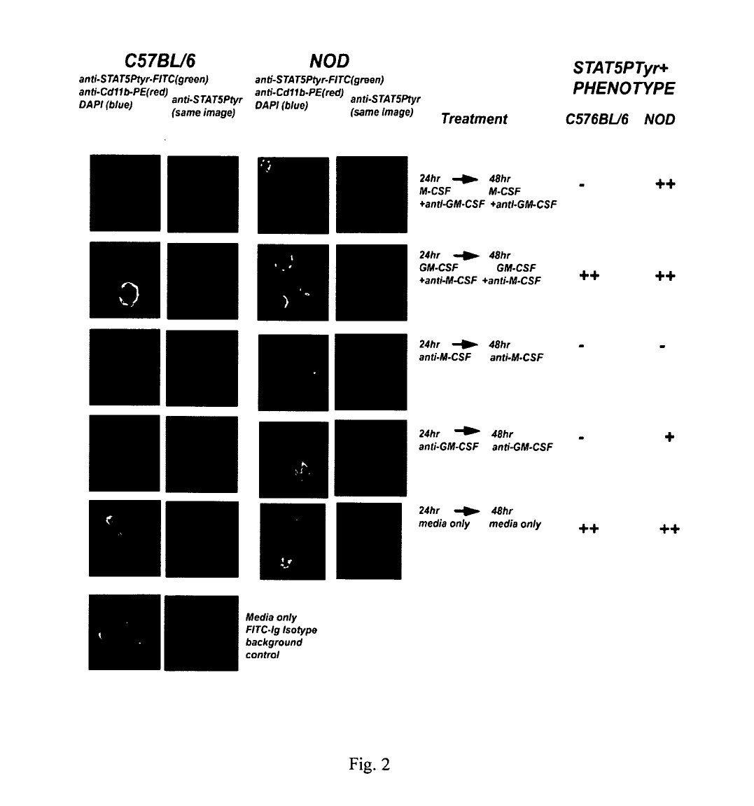

Deconvolution microscopic image analysis (FIG. 2) showed that the NOD STAT5 phosphorylation phenotype could be created in non-autoimmune C57BL/6 cells by increasing the GM-CSF stimulation to the level seen in the NOD. In addition, treatment with M-CSF prior to GM-CSF also diminished the amount of STAT5 phosphorylation detectable in these projections.

Blocking GM-CSF signaling by anti-GM-CSF antibodies or with the Jak2/3 inhibitor AG490 in mature monocytes and macrophages does not affect the persistence of phosphorylated STAT5 [20]. However, STAT5 phosphorylation can be diminished in both NOD bone marrow cells by blocking endogenous GM-CSF or M-CSF stimulation (FIGS. 2 and 3). Furthermore, flow cytometric analysis indicated that blocking either endogenous GM-CSF while stimulating with M-CSF or endogenous M-CSF while stimulating with GM-CSF promoted STAT5 phosphorylation in both NOD and C57BL/6 48 hr bone marrow cultures. When bone marrow cultures were stimulated for 48 hr with M-CSF in the presence of endogenous GM-CSF, STAT5 phosphorylation were represented in C57BL/6 cultures, but accentuated in NOD (FIG. 3).

When we sequentially blocked M-CSF and stimulated with GM-CSF then blocked GM-CSF while stimulating with M-CSF in these cultures, we found that C57BL/6 cells suppressed STAT5 phosphorylation and NOD cells accumulated phosphorylated STAT5 (FIG. 4). Reversing the order of cytokines and blocking antibodies to mirror the natural order in vivo; i.e., GM-CSF with blocking M-CSF for 24 hr then M-CSF while blocking GM-CSF for the next 24 hours, reversed the effects on STAT5 phosphorylation (FIG. 4).

Taken together, these data support the interplay of temporally sequenced GM-CSF and M-CSF signaling on STAT5 activation during myeloid cell differentiation. This interplay caused by the timing and concentration of GM-CSF stimulation early in differentiation sets the stage for STAT5 activation in later stages, regardless of further stimulation of GM-CSF or M-CSF on the cells.

To examine the STAT5 isoforms and their functionality involved in this GM-CSF to M-CSF, DNA binding affinity precipitation (DAP) and EMSA experiments on our in vitro differentiation cultures (FIG. 5). Previous studies in primary monocyte and macrophage cultures indicated that GM-CSF-induced STAT5 function in autoimmune myeloid cells changed between monocytes and macrophages [20,23]. Each differentiation stage had its own unique pattern of STAT5 isoforms and DNA binding function. NOD monocytes had an apparent increase in phosphorylated truncated STAT5, but these isoforms were unable to bind DNA when stimulated with GM-CSF, similar to STAT5 in autoimmune human subject monocytes. In contrast, GM-CSF-activated STAT5 in NOD unelicited peritoneal macrophages stimulated phosphorylation and nuclear localization of full-length STAT5A & B isoforms in addition to the high levels of cytoplasmic truncated STAT5 isoforms seen in their monocytes.

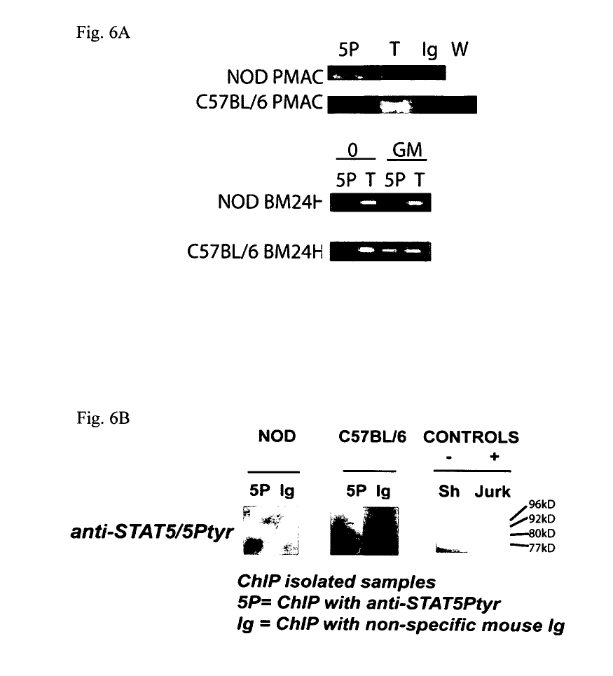

The activated full-length isoforms of STAT5 had persistent DNA binding on GAS (gamma-activated sequences, STAT5 binding sites) binding sequences in DAP and EMSA experiments compared to C57BL/6 control strain macrophages[20]. When DAP and ChIP analysis was run on GM-CSF-stimulated bone marrow-derived myeloid cell cultures, distinct changes in STAT5 binding were observed at 24 h and 48 h in culture (FIGS. 5 and 6).

In NOD cultures grown 2 days in the presence of M-CSF and high endogenous GM-CSF levels, truncated STAT5 isoforms (77kD) were the primary isoforms found in the nuclear fraction. These proteins were activated but remained unbound to GAS DNA sequences. At 4 days in M-CSF, NOD cells had activated full-length STAT5 and truncated isoforms present, still unbound to DNA, and mainly found in the cytoplasmic fraction. In contrast, C57BL/6 control bone marrow cultures similarly treated 2 days with M-CSF had both activated full-length and truncated (80k) in their nuclear fractions. Though both isoforms were able bind to GAS sequences, the majority of DNA bound STAT5 found in the nuclear fraction of the C57BL/6 cells was full-length activator STAT5. After 4 days, the C57BL/6 cultures had low endogenous GM-CSF levels and the activated STAT5 isoforms in C57BL/6 nuclear fractions lost their DNA binding capacity. The small amount of truncated' isoforms found in these cultures were unactivated and only in the cytoplasmic fraction. These findings suggest a change, in GM-CSF expression coincides with a change in STAT5 isoform activation and DNA binding.

Our data from NOD bone marrow and mature myeloid cells indicate that overexpression of GM-CSF in these cells prolongs the activation of truncated STAT5 isoforms which have lost their DNA binding capacity.

### EXAMPLE 2

GM-CSF expression by, and autocrine stimulation of, myeloid cells is a key temporal regulatory component in myeloid differentiation and activation responsiveness in mature monocytes and macrophages. GM-CSF gene expression can be induced in myeloid cells by cytokines such as IL-6 and IL-3, which release epigenetic control blocks on its promoter. The mechanism of this control is through the interaction of acetylases and deacetylases at specific regions within the Csf2 gene promoter, which can be STAT protein mediated through its role as an adaptor protein for these enzymes. Full-length isoforms of STAT5 can act as adaptors for both acetylases and deacetylases [27,28]. Truncated STAT5 acts as an adaptor for deacetylases only, having post-translationally lost its COOH terminal binding site for acetylases.

To examine whether STAT5 can be involved in the epigenetic regulation of the Csf2 gene during myeloid differentiation, the STAT5 isoforms bound to the Csf2 gene promoter at different stages of myeloid differentiation were captured. Using CUP analysis, STAT5 binding at the Csf2 promoter in mature macrophages and bone marrow derived myeloid cells during in vitro differentiation (FIG. 6) were examined. It was found that STAT5 can bind to the Csf2 promoter region in mature NOD macrophages and not in C57BL/6 without any activation. STAT5 is found bound to this region in untreated bone marrow cultures of both strains (FIG. 6) but this interaction may not be specific to myeloid cell precursors in the sample. In stark contrast, NOD bone marrow cell STAT5 loses its binding capacity when the cells are differentiated by GM-CSF, while its bind in similarly treated C57BL/6 bone marrow cells is greatly enhanced. Though immunoprecipitation analysis of STAT5 in bone marrow cultures showed the presence of both full-length and truncated STAT5 isoforms in both NOD and C57BL/6 myeloid cells (FIG. 5b), western blot analysis of anti-STAT5Ptyr chromatin precipitated protein (FIG. 6b) and DAP-isolated STAT5Ptyr (FIG. 5a) indicate that only truncated isoforms are found strongly activated and bound to DNA in unactivated and GM-CSF stimulated non-autoimmune myeloid cells.

In comparison, NOD myeloid cells showed only full-length isoforms activated in the nucleus (FIG. 5b), which may possibly able to associate with DNA/chromatin in untreated myeloid cells (FIGS. 6b and 5a). The highly phosphorylated truncated STAT5 isoforms in NOD cells are found mainly in the cytosolic fraction and unable to bind DNA in either assay.

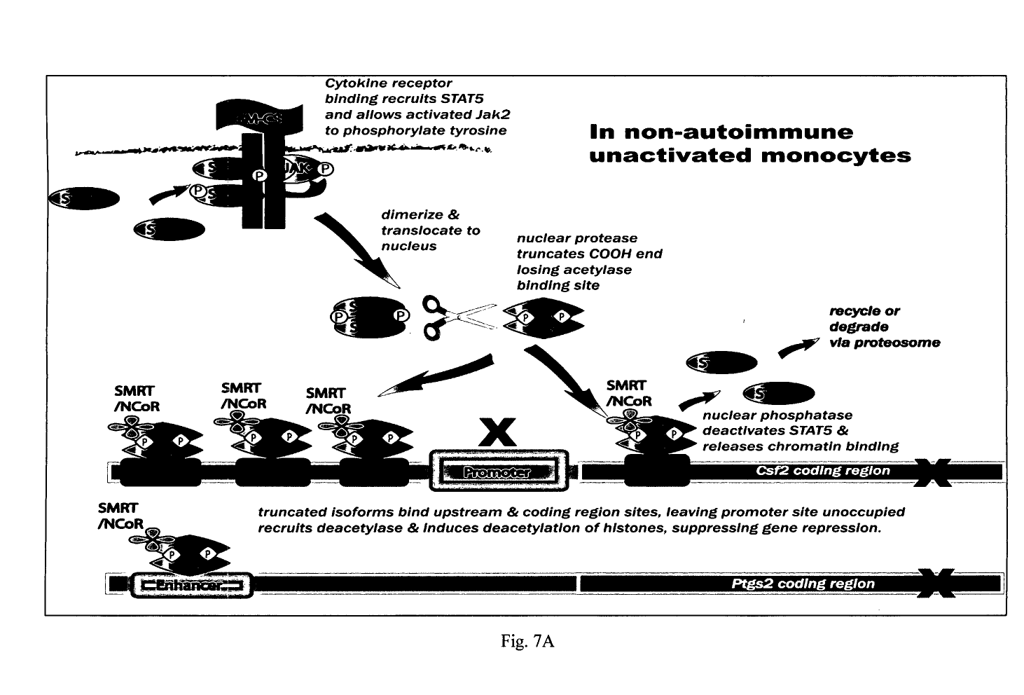

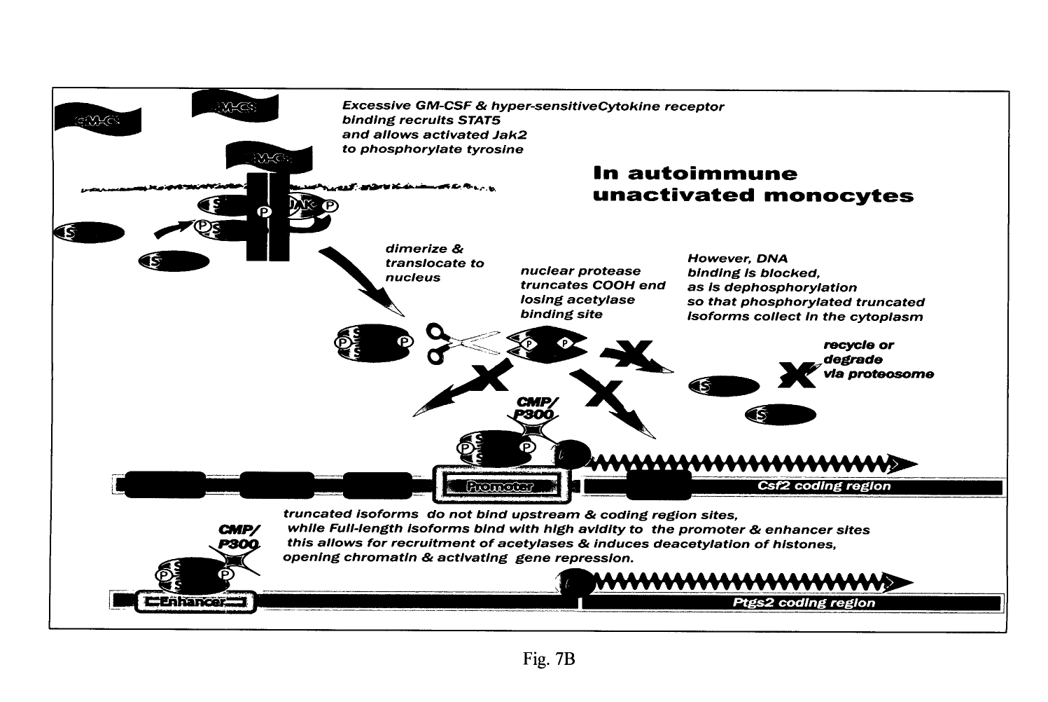

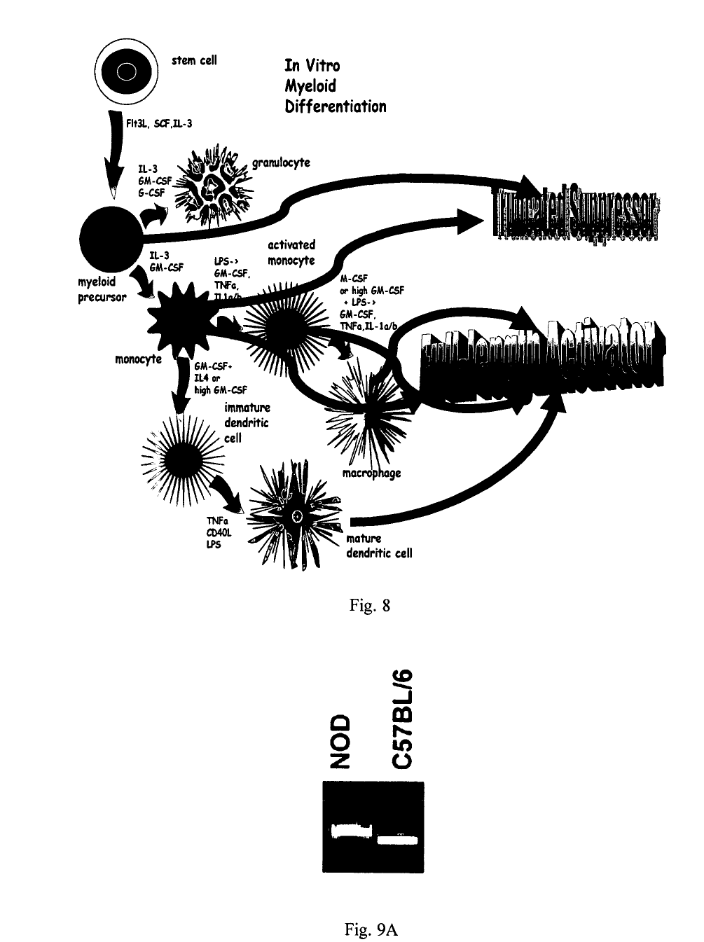

These data help us to resolve the seemingly paradoxical link between persistent STAT5 phosphorylation in autoimmune macrophages when GM-CSF or Jak 2 signaling was blocked, while the reverse was true in bone marrow cultures [20]. In mature cells, GM-CSF promotes full-length STAT5 isoform binding on DNA but its own gene expression is not dependent on this activation. However, during differentiation, where GM-CSF temporal regulation is critical to the successful completion of the maturation process, the loss of gene suppression by STAT5 truncated isoform binding at the Csf2 promoter allows for precocious and prolonged expression of GM-CSF, which interferes with the ability of the cell to respond properly to subsequent M-CSF driven maturation signals. Therefore, we formulated a mechanistic model (FIG. 7) for STAT5's role in the dysregulation of Csf2, and possibly other GM-CSF regulated genes such as PGS2/COX2. We hypothesized that myeloid differentiation activation of GM-CSF sets up the initial activation of STAT5 which is then perpetuated its own activation and GM-CSF overexpression through STAT5 dysregulation of the Csf2 and/or other genes within the NOD Idd4.3 locus. Thus, the loss of an autocrine feedback regulation of its own gene, through its own upregulation STAT5 binding or another STAT5-activating gene encoded within the Idd4.3 locus, GM-CSF gets overexpressed, blocking STAT5 regulation by M-CSF and IL-10, and disrupting M-CSF signaling to promote completion of myeloid cell differentiation.

Our data indicate that STAT5 can bind to sequences in the Idd4.3 region in mature but not immature NOD myeloid cells, in direct contrast to its binding character in non-autoimmune mouse myeloid cells. Knowing that STAT5 binding in vivo promotes gene regulation at the epigenetic not genetic level [27,28], our ChIP analysis of STAT5 binding in vivo on chromatin rather than naked DNA as in EMSA or DAP, indicates a change in the epigenetic modification of histones within the non-coding chromatin upstream of the Csf2 gene is lost in NOD bone marrow derived myeloid cells: but gained in mature NOD macrophages. Thus, too much GM-CSF for too long during myeloid differentiation blocks subsequent signaling from M-CSF necessary for the completion of the maturation process.

The autoimmune STAT5 dysfunction seen in the NOD may be best described as a ‘broken switch’ in cytokine signaling during myeloid differentiation, shorting out the normal activation and turn-over sequence of cytokines influencing precursor cells on their way: to maturation. The incompletion of myeloid cell maturation blocks the eventual functionality of this important class of APC in the initiation and maintenance of self-tolerance leading to autoimmunity.

### Additional Methods and Materials

### Mouse Strains

Five to twelve week old NOD and C57BL/6 female mice (The Jackson Laboratory, Bar Harbor, Me.) were used for these studies. At least 2 mice of each strain were used for tissues in each run of the experiment and each analysis was run at minimum in triplicate.

### Bone Marrow In Vitro Differentiation & Sample Preparation

Mice were euthanized and the long bones of the hind limbs were excised. Bone marrow cells were flushed out of the bones using a 30-guage needle and syringe filled with ice cold RPMI medium supplemented with 10% fetal calf serum and 1% antibiotic/antimycotic mix (Cellgro-Mediatech, Herndon, Va.). The marrow cells were washed with cold media then the red blood cells in samples were lysed by incubation in sterile cold 0.84% NH4Cl buffer. The remaining bone marrow cells were then plated on tissue culture dishes and fed with fresh, sterile medium alone or with 1000 U/ml of GM-CSF (Biosource/Invitrogen, Carlsbad, Calif.) then followed 30 min after with the addition or omission of 100 μM Na vanadate in DMSO (Sigma-Aldrich, St Louis, Mo.) to the culture medium. Cultures were maintained for 2 (with and without Na vanadate) or 24 hr (without Na vanadate) at 37 C/5% CO2. An aliquot of cells was taken to confirm phenotypic identification and phosphotyrosine STAT5 analysis by flow cytometry as previously described [20, 23]. After incubation, half of the media volume from these cultures was collected and frozen at −80 C to confirm GM-CSF concentration by Luminex (Upstate Biotech Beadlyte kits, Upstate USA, Millipore, Chicago, Ill.) and ELISA (BD Biosciences OptiEIA kits, San Diego, Calif.) as previously described [19, 20, 23]. Cells were fixed in situ with 1% (v/v Cf) formaldehyde (methanol-free, Sigma-Aldrich) added in the remaining media for 10 min at 37 C, then washed with 1×PBS, and sonicated in SDS Lysis Buffer (Upstate)+protease inhibitors (Roche, Indianapolis, Ind.) for later analysis in chromatin immunoprecipitation (ChIP).

### Monocyte and Macrophage Collection & Culture

Mice were euthanized and approximate 50 μl of blood collected via cardiac puncture post mortem. Blood was immediately processed for analysis of STAT5 phosphorylation by flow cytometry as previously described for NOD myeloid cells [14]. After blood collection, the peritoneal cavity was filled with ice cold RPMI medium supplemented with 10% fetal calf serum and 1% antibiotic/antimycotic mix (Cellgro-Mediatech, Herndon, Va.) using a 20-gauge needle and syringe. The lavage fluid and cells were withdrawn and washed with cold media by centrifugation. Liver tissue was collected after lavage for use in genomic DNA preparation. Red blood cells in lavage samples were lysed by incubation in sterile cold 0.84% NH4Cl buffer. The remaining cells were then plated on tissue culture dishes for 1 hour at 37 C/5% CO2. The non-adherent cells were washed off the plates with sterile 1×PBS (Cellgro-Mediatech) and fed the adherent macrophage re-fed with fresh sterile medium alone or with 1 000 U/ml of GM-C SF (Biosource/Invitrogen) for future ex vivo culture at 37 C/5% CO2. An aliquot of cells was taken to confirm phenotypic identification and phosphotyrosine STAT5 analysis by flow cytometry as previously described [13,14]. After incubation, half of the media volume from these cultures was collected and frozen at −80 C to confirm GM-C SF concentration by Luminex (Upstate) and ELISA (BD Biosciences) as previously described [12-14]. Cells were fixed in situ with 1% (v/v Cf) formaldehyde (methanol-free, Sigma-Aldrich) added in the remaining media for 10 min at 37 C, then washed with 1×PBS, and sonicated in SDS Lysis Buffer (Upstate)+protease inhibitors (Roche) for later analysis in chromatin immunoprecipitation (ChIP).

### Sequence Analysis of Csf2 Gene Promoter

Genomic DNA from each strain was prepared from liver and amplified in PCR using Roche Biosciences Master Mix reagents and primers (3′-3 bp: CTA AAG CAT GTT TCT TGG CTA; 3′-350 bp: AGA AGC AGT TCC TGA TTC CA; 3′-642 bp: AAA GAG GCT CAC ATA ACT CA; 5′-969 bp: AAA TAA GGT CCA GCC CAA TG; Integrated DNA Technologies, Coralville, Iowa) designed to amplify regions in the −3 to −969 bp sequence upstream of the Csf2 gene. The amplified DNA was gel purified using Qiagen gel extraction reagents and phenol/chloroform extracted (Qiagen, Valencia, Calif.). The amplified fragment was then used as template in a Big Dye PCR amplification reaction (Applied Biosystems) and sequenced using an AB capillary sequence analyzer (Applied Biosystems). ChromasLite and ClustalW freeware were used for the sequence analysis and alignment. Analysis of secondary structure in amplicon primers and sequence regions was analyzed using Oligo 4.0 primer analysis software copyrighted by Wojciech Rychlik.

### Chromatin Immunoprecipitation (ChIP) Analysis of STAT5 Binding at the Csf2 Promoter

Four million cells from bone marrow cultures or ex vivo peritoneal macrophages were fixed in situ with 1% formaldehyde in 1×PBS (methanol-free, Sigma-Aldrich) for 10 min at 37 C prior to lysis with High SDS Lysis Buffer (Upstate). The fixed lysates were sonicated to disrupt membranes and shear chromatin to approximately 1000 bp fragments then frozen until analysis. Once thawed, the samples were divided into aliquots for each run of the analysis. The aliquots used for IP were pre-cleared with salmon sperm DNA Protein A or G agarose beads (Upstate), then incubated overnight at 4 C with anti-tyrosine phosphorylated STAT5 (STAT5Ptyr) antibodies (Upstate). After incubation, the antibody-bound chromatin complexes were recipitated using salmon sperm DNA Protein A agarose beads, and washed extensively with a series of increasing stringency buffers (low salt, high salt, LiCl, TE; ChIP reagent kit, Upstate). A non-specific antibody control (mouse IgG, UpState) and a no extract sham IP were run as negative controls.

Total cell and ChIP extract aliquots were dissociated from the beads in 1% SDS, 0.1M Bicarbonate buffer (Fisher Scientific, Atlanta, Ga.). NaCl was then added to a final concentration of 500 mM and the samples incubated 4 hr at 65 C to reverse the formaldehyde crosslinks. DNA was purified from these aliquots for PCR and real time PCR amplification of DNA sequences from Csf2 promoter which have been shown to be epigenetic regulatory sites for inducible Csf2 expression [24, 25].

ChIP isolated DNA samples were volume matched in all PCR runs to 100 ng of total DNA extracts from same samples. Standard PCR was run using Eppendorf Master 2.5× Mix (Fisher Scientific) and a cycle protocol of 94 2 min, 94 C 30 sec, 55-60 C annealing temperature (dependent on the primer set used) 30 sec, 72 C 30 sec, for 35 cycles. Products of the amplification were separated in a 3% agarose gel (SeaKem, Fisher Scientific) and visualized by ethidium bromide (Fisher Scientific) intercalation. Real time PCR was run using Sybr Green Master Mixes (Applied Biosystems, Foster City, Calif. or BioRad, Hercules, Calif.) either with the same protocol as described for standard PCR above or in a 98 C hot-start protocol designed to remove secondary structure in the DNA template. Due to the pallindromic nature of STAT5 binding sites, DNA containing these regions forms complex secondary structure which can block polymerization in vitro, giving false negative results in standard PCR-based DNA binding assays. To resolve these structures for analysis, we modified our PCR protocol to include the addition of 2%-5% DMSO (Sigma-Aldrich) in the reaction mix and a cycle profile of 98 C 5 min, 94 C 30 sec, 55-60 C (dependent on the primer set used) 30 sec, 72 C 30 sec, for 45 cycles. Real time amplification quantitation was compared on the basis of R values calculated as R=2^{((Nonspecific Ig ChIP Ct)-(anti-STAT5Ptyr ChIP Ct))}[29]. Statistical analyses of data were performed using Prism 4 software (GraphPad, San Diego, Calif.).

For ‘double ChIP’ (db ChIP) protein analysis, the anti-STAT5 selected complexes were washed extensively and eluted from the beads as described above for ChIP DNA sample isolations, but were then neutralized and re-precipitated overnight at 4 C using pre-coupled anti-histone 113-Protein G Salmon sperm DNA coated beads. The db ChIP complexes were then washed as before and then boiled in Leammili buffer for western blot analysis. Western blots were probed with anti-STAT5 Ptyr (UpState) antibodies and crosslinked and uncoupled proteins were visualized using Amersham ECL plus chemilumenescence reagents (GE Healthcare/Amersham, Piscataway, N.J.). Blots were stripped and re-probed with antibodies to acetylated histone 113, RNA polymerase II, SMRT, and P300 (Upstate). Densitometry analysis using BioRad Imager and Quantity One Software was used to estimate molecular weight of cross-linked dimer complexes and freed monomeric isoforms in STAT5Ptyr positive bands.

### EXAMPLE 3

### NOD Csf2 Promoter Region Contains a Unique Microsateiite DNA Insertion

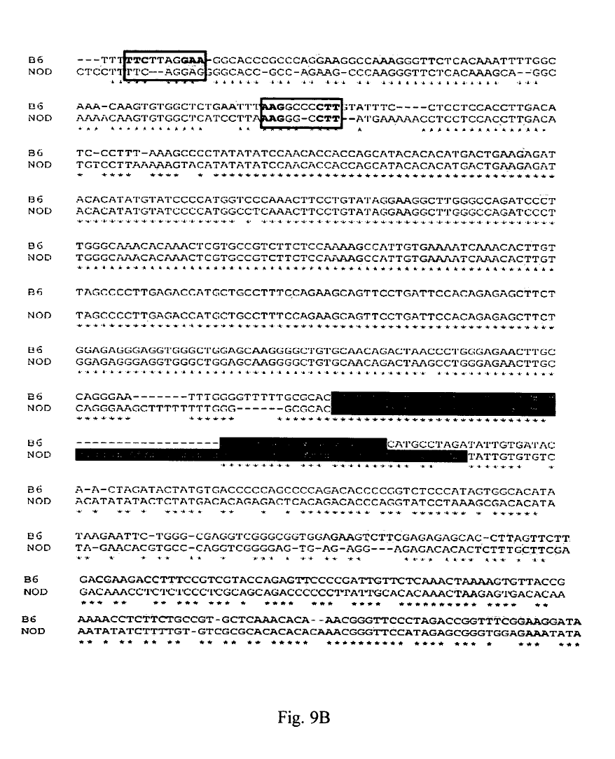

Comparative sequence analysis of the region 1 kb upstream of the Csf2 gene in NOD and non-autoimmune C57BL/6 mice revealed a microsatellite DNA insertion in the NOD not found in the control strain (FIG. 9). This 1 kb region also contained at least 2 potential STAT protein GAS (gamma activation sequences) binding sites (TTCNNNGAA/AAGNNNCTT) [30, 31], in both 5′ to 3′ and 3′ to 5′ orientations. There are also several ‘half’ or imperfect STAT5 binding sites within this region. Such suboptimal sites have the potential for binding STAT proteins as part of multimeric binding facilitated by STAT protein bound at a nearby GAS site [30, 31]. At least 2 potential STAT5 binding sites (underlined in FIG. 9) within this region are altered in the NOD as compared with C57BL/6; one lost (5′->3′) and one gained (3′->5′). At the 3′ to 5′ site, C57BL/6 mice have a GAS site that can bind either STAT6 or STAT5, with preference to STAT6; whereas, the NOD sequence at this region contains a strong STAT5 binding site that would not support STAT6 binding [30-32].

### EXAMPLE 4

### GM-CSF Mediates Increased STAT5 Binding on Chromatin in NOD Bone Marrow Cells

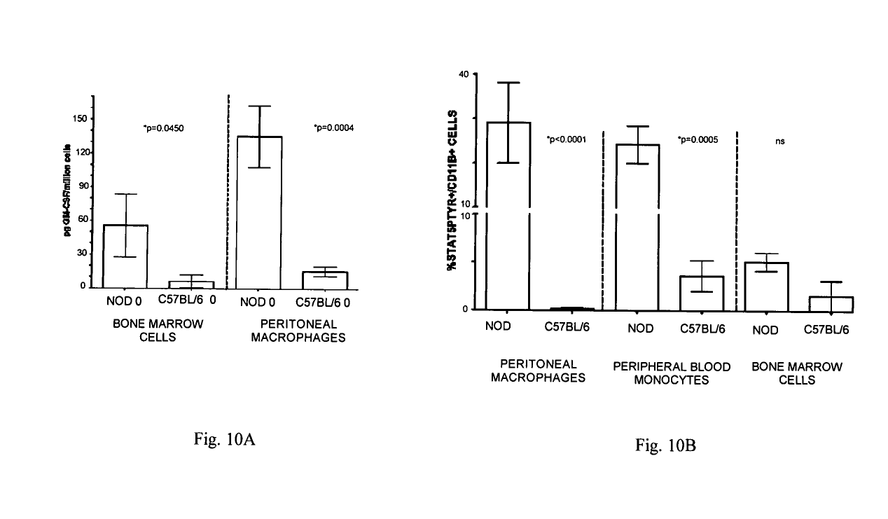

Like NOD macrophage and monocytes, NOD bone marrow cells have increased GM-CSF expression and high STAT5 phosphorylation compared to C57BL/6 mouse bone marrow cells (FIG. 10).

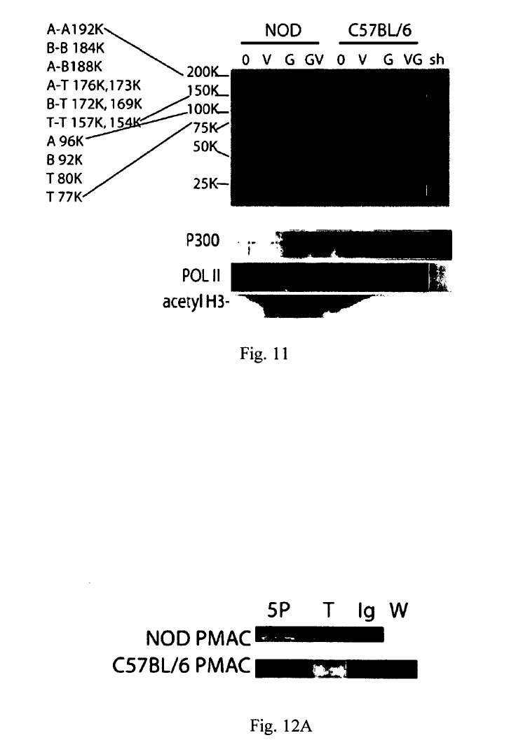

We performed double ChIP analyses on NOD and C57BL/6 bone marrow cells that had been cultured with GM-CSF for only 2 hr, and looked for in situ assembly of chromatin-associated protein complexes which containing both STAT5 and Histone H3 (FIG. 11). We controlled for the possibility of the prolonged phosphorylation of cytoplasmic DNA-unbound STAT5 masking a more transient phosphorylation of DNA-bound STAT5 isoforms, by culturing these cells in the presence and absence of the phosphatase inhibitor, sodium vanadate. In these dbChIP analyses, high molecular weight bands containing phosphorylated STAT5 corresponding to phosphorylated STAT5 monomers were detected only in GM-CSF-treated NOD cells (FIG. 11). Faint but reproducible bands for P300, RNA polymerase II, and acetylated Histone H3 were also found only in GM-CSF treated NOD cell dbChIP isolates. Complexes of SMRT-STAT5Ptyr-Histone H3 proteins were not detected in any of the dbChIP isolates. Higher molecular weight STAT5-containing complexes were detected in both NOD and C57BL/6 cell dbChIP isolates, though at markedly different levels. Moreover, the level of these complexes in C57BL/6 dbChIP isolates did not significantly change with GM-CSF and/or vanadate treatment. These higher molecular weight bands may be an artifact or represent complexes containing formaldehyde crosslinked dimers of STAT5 in multiple size isoforms (i.e., STAT5A and STAT5B homo- and hetero-dimers, either in full-length or truncated isoforms). Densitometry estimates of such isoform complexes and their sizes are given on the left side of the blot in FIG. 11, top panel). No bands at any of the sizes predicted for monomeric STAT5A, STAT5B or truncated isoforms were seen associated with histone H3 in any of the treatments used in C57BL/6 cell cultures. These findings suggest that at least transient binding of full-length isoforms of STAT5 on chromatin is inducible in NOD but not C57BL/6 bone marrow cells treated with GM-CSF.

GM-CSF-Induced STA T5 Chromatin binding at Epigenetic Regulatory Sites within the Csf2 Promoter

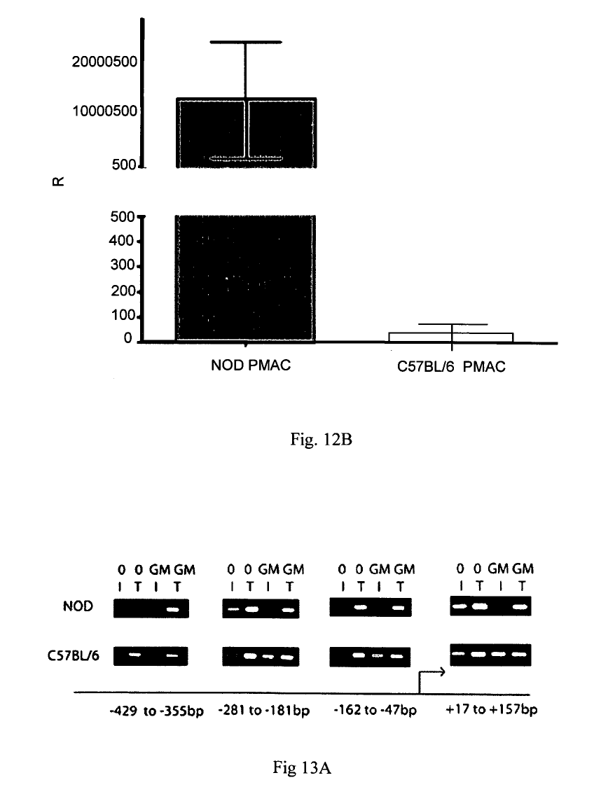

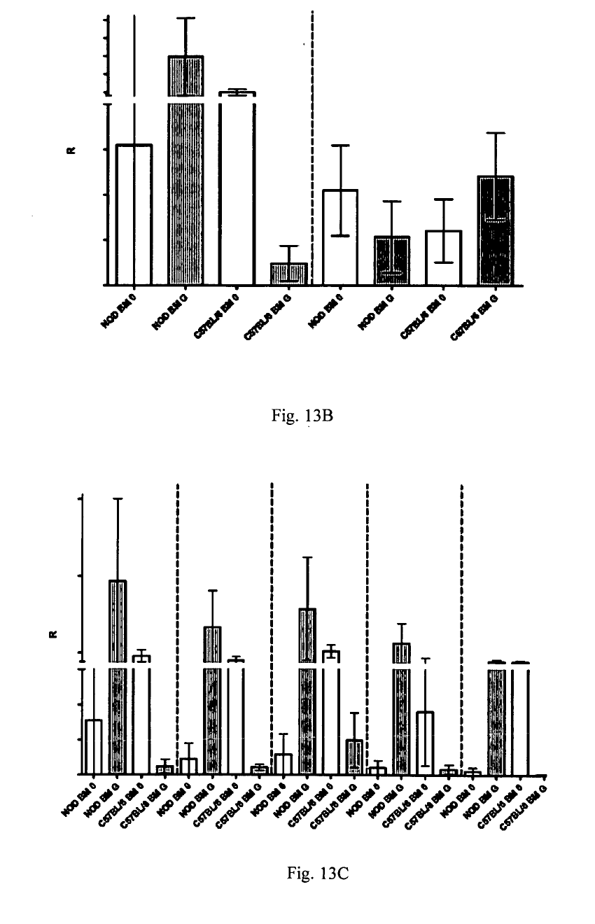

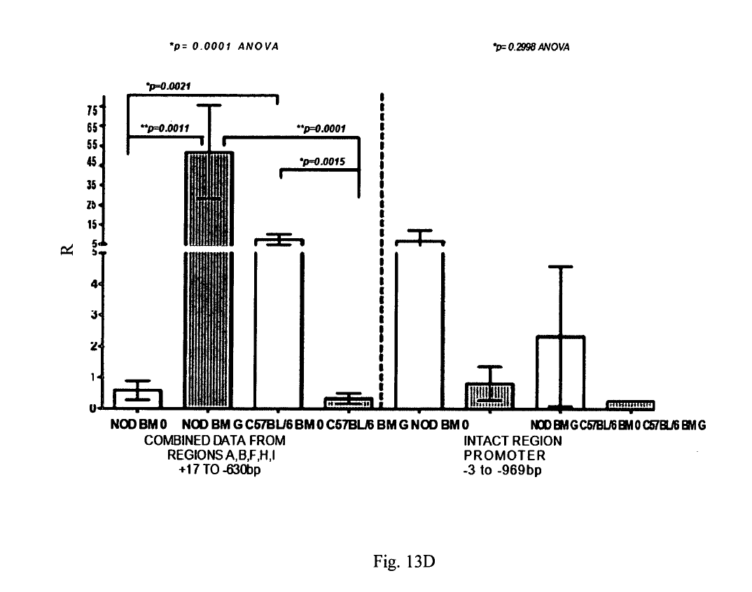

To test for the possible involvement of STAT5 in regulation of Csf2, STAT5Ptyr-mediated ChIP were used to isolate DNA for use as templates in PCR with primers detect identified deacetylase binding sites within the first 1000 bp of the promoter region upstream of the Csf2 gene coding sequence [24, 25]. STAT5 proteins in NOD peritoneal macrophages exhibit strong binding on sequences immediately upstream of the Csf2 gene (−181 bp to +10 bp [24]), without exogenous GM-C SF stimulation; whereas, this site was not found bound to STAT5 in C57BL/6 cells (FIG. 12). In contrast, standard PCR analyses of anti-STAT5Ptyr-mediated ChIP isolated DNA showed distinct and reproducible GM-CSF-induced changes in STAT5 binding at several of these potential epigenetic modification sites within or near the Csf2 promoter region (FIGS. 13a and 13b). The loss of STAT5 binding after 24 hr of GM-CSF stimulation in NOD bone-marrow cells at sites in the −47 to −162 bp, −181 to −281 bp, and +17 to +157 bp regions of the Csf2 promoter contrasted sharply with the GM-CSF-induced increase in STAT5 binding seen at these sites in C57BL/6 cells, and with the data in our dbChIP analyses which indicated the opposite STAT5 binding capabilities induced by GM-CSF (FIG. 11).

### EXAMPLE 5

### GM-CSF-Induced STA T5 Chromatin Binding within the Csf2 Promoter Involves DNA Secondary Structure in the DNA Binding Region

Sequence analysis using secondary structure modeling software suggested that the STAT5 binding sites within the Csf2 promoter region had very high potentials for internal self-annealing and; thus, were highly likely to have intense secondary structure that could block efficient polymerase amplification of the sequence. Therefore, we repeated our ChIP PCR analyses using a modification of the PCR protocol to remove secondary template/primer structure prior to polymerization. In these modified real time PCR protocols, we uncovered a marked increase in the presence of Csf2 promoter DNA in anti-STAT5Pytr-mediated ChIP DNA isolates in GM-CSF activated NOD bone marrow cells when compared to C57BL/6 bone marrow cell samples or with unactivated NOD bone marrow cells (FIG. 13b-d).

When STAT5 binding within the entire −3 to −969 bp region upstream of the Csf2 transcriptional start site was analyzed, DNA from anti-STAT5Ptyr bound complexes from both GM-CSF-treated NOD and C57BL/6 bone marrow cells still showed an overall decrease in STAT5 binding compared with untreated cultures (FIG. 13d). One possible explanation for these finding is that cells other than myeloid precursors are surviving in the untreated mixed bone marrow cultures and may contribute to apparent level of STAT5Ptyr binding seen in these isolates. However, combined analyses of STAT5 binding at all individual epigenetic regulatory regions tested showed a marked enhancement of STAT5 with GM-CSF stimulation in NOD bone marrow cells at these sites as compared with C57BL/6 (FIG. 13d).

This suggests that GM-CSF is actually enhancing STAT5 binding at specific sites within its own gene's promoter in NOD, while reducing it in C57BL/6 bone marrow cells. Analysis of individual epigenetic regulatory sites within the Csf2 promoter indicates that GM-C SF modulates STAT5 binding differently at each site tested (FIG. 13c), but does not clearly define whether STAT5 binding at each site is independent, synergistic, or antagonistic to each of the other sites.

Our ChIP real time DNA PCR and double ChIP protein analyses together suggest that GM-C SF induces full-length STAT5 binding at the Csf2 promoter site in NOD mouse bone marrow cells but not in the non-autoimmune C57BL/6 mouse cells. GMCSF induced STAT5 binding on sites within the Csf2 promoter may reflect GM-CSF's ability to regulate its own expression through a positive feedback loop mediated through STAT5. Such a regulatory mechanism would have the potential to increase histone acetylation within the Csf2 promoter, through the recruitment of CBP/P300, resulting in epigenetic dysregulation of this chromosomal region, and promote the overexpression of GM-CSF seen in NOD myeloid cells.

As a consequence of prolonged GM-CSF production in these autoimmune cells, the temporal window for controlling GM-CSF's influence on myeloid differentiation and in inflammatory responses may be overridden. Loss of this window of GM-CSF independence in myeloid differentiation leads to loss of subsequent responsiveness to M-CSF and adequate myeloid APC maturation, as seen in the NOD.

The mouse Csf2 promoter region has an overall 61% homology with human Csf2 promoter, and even higher homology in the specific epigenetic regulatory regions focused on in these studies [24, 25]. Thus, polymorphisms within the Csf2 promoter may provide evidence for a genetic component underlying the aberrant GM-C SF and STAT5-phenotypes which NOD myeloid cells share with human autoimmune myeloid cells. Preliminary data in congenics further suggest that STAT5 binding to epigenetic regulatory sites within the Csf2 promoter impacts not only GM-CSF expression, but GM-CSF induction of persistent STAT5 activation and STAT5-mediated dysregulation of Ptgs2. Thus, the dysregulation of Csf2 may be indicative of more widespread effects on the expression of other genes regulated by GM-CSF through STAT5.

### EXAMPLE 6

### Diagnostic Procedures

Fresh blood samples can be collected by venipuncture or finger/heel prick in accordance with standard clinical practices. Samples will typically be useful if received for analysis within 48 hours of collection and kept at 4 C until analysis. One hundred microliters (100 μl) to 1 milliliter (ml) of whole blood can be collected into heparinized blood vials at each sample drawings. Samples can be separated by centrifugation (600×g, 5 min., 20 C) for plasma collection. After the plasma layer is removed and frozen for GM-CSF Luminex and PGE2 ELISA analyses, the cells will be resuspended in RPMI+10% FCS for culture or analysis.

### PGS2/COX2, STAT5Ptyr, & CD14 Fluorescent Immunohistochemistry & Flow Cytometric Analysis:

Flow cytometric analysis of fluorescently-conjugated antibody surface and intracellular binding can be used to phenotypically identify myeloid cells, and quantify the levels of PGS2/COX2 protein, and STAT5 tyrosine phosphorylation in cells from whole blood samples. Cells can be first labeled with anti-CD14 to identify monocytes (CD14+) in the sample, then fixed with 2% (v/v C_{f}) formaldehyde and permeabilized prior to intracellular staining with anti-STAT5Ptyr, PGS2/COX2, and/or GM-CSF specific antibody fluorescent conjugates. These methods have shown good consistency.

PTGS2 enhancer/CSF2 promoter Polymorphism Genotype Analysis:

Genomic DNA from each sample can amplified via PCR using Roche Bioscience Master Mix reagents and primers (e.g., 5′ CTA AAG CAT GTT TCT TGG CTA; 3′ AAA TAAA GGT CCA GCC CAA TG; Integrated DNA Technologies, Coralville, Iowa[24, 25]) designed to amplify the −3 to −969 bp sequence upstream of the Csf2 gene, and primers (e.g., 5′-TTCCGGGAA; [5, 30].) designed to amplify the −1000 bp to +10 bp region upstream of the Ptgs2 gene. The amplified fragment cab be used as template in a Big Dye (Applied Biosystems, Foster City, Calif.) reaction and sequenced using a calibrated AB capillary sequence analyzer (Applied Biosystem). ChromasLite and ClustalW freeware can be used for the sequence analysis and alignment. NIH Aligner Software can be used to align and compare control and subject DNA sequences for polymorphisms statistically unique to the T1D/at-risk population. All sequences can be confirmed in both 5′ and 3′ directed amplification, each run in triplicate. Known sequence standard DNA for both control and T1D subject sequences will be included in each analysis run.

### Prostaglandin Profile Analysis:

ELISA analysis can be used to measure the prostaglandin production from PBMC samples. EIA kits for mot prostaglandin products of interest are commercially available from Cayman Chemical Company.

### GM-CSF, PGE2, and Other Secreted Product Analyses:

Multiplex Luminex analysis kits can be used to quantify monocyte GMCSF, IL10, IL12, TNFα, and ILβ in plasma from cell samples. In addition, Luminex assays are purchased as kits or bead prep systems for developing PGE2, STAT5Ptyr, and PGS2/COX2 Luminex assays from commercial sources, Linco, UpState (Millipore) and BioRad. Antibodies for biomarker detection on beads or plates in these assays can be purchased from Endogen (GM-CSF), Upstate (STAT5Ptyr), and Cayman (PGS2/COX2 and PGE2).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview, of this application.

### REFERENCES

1) Teglund, S., C. McKay, E. Schuetz, J. M. van Deursen, D. Stravopodis, D. Wang, M. Brown, S. Bodner, G. Grosveld, and J. N. Ihle. 1998. Stat5a and Stat5b proteins have essential and nonessential, or redundant, roles in cytokine responses. 1998. Cell. 93: 841-850.
2) Hamilton, J. A. 2002. GM-CSF in inflammation and autoimmunity. Trends in Immunol. 23(8): 403-408.
3) Hashimoto, S., I. Komuro, M. Yamada, and K. S. Akagawa. 2001. IL-10 inhibits granulocyte-macrophage colony-stimulating factor-dependent human monocyte survival at the early stage of the culture and inhibits the generation of macrophages. J. Immunol. 167: 3619-3625.
4) Piazza, F., J. Vlens, E. Lagasse, and C. Schindler. 2000. Myeloid differentiation of FdCP1 cells is dependent on Stat5 processing. Blood. 96(4): 1358-1365.
5) Yamaoka, K. T. Otsuka, H. Niiro, Y. Arinobu, Y. Nihho, N. Hamasaki, and K. Izuhara: 1998. Activation of STAT5 by lipopolysaccharide through granulocyte-macrophage colony-stimulating factor production in human monocytes. J. Immunol. 160: 838-845.
6) Azam, M., C. Lee, I. Strehlow, and C. Schindler. 1997. Functionally Distinct Isoforms of STAT5 are generated by Protein Processing. Immunity. 6: 691-701.
7) Nakajima, H., P. K. Brindle, M. Handa, and J. N. Ihle. 2001. Functional interaction of STAT5 and nuclear receptor co-repressor SMRT: implications in negative regulation of STAT5-dependent transcription. 2001. EMBO J. 20(23)L 6836-6844.
8) Lee, C., F. Piazza, S. Brutsaert, J. Valens, I Strehlow, M. Jarosinski, C. Saris, and C. Schindler. 1999. Characterization of the Stat5 Protease. J. Biol. Chem. 274(38): 26767-26775.
9) Azam, M., H. Erdjument-Bromage, B. Kreider, M. Xia, F. Quelle, R. Basu, C. Saris, P. Tempst, J. N. Ihle, and C. Schindler. 1995. Interleukin-3 signals through multiple isoforms of Stat5. EMBO J. 14(7): 1402-1411.
10) Novak, U., A. Mui, A. Miyajima, and L. Paradiso. 1996. Formation of STAT5-containing DNA binding complexes in response to Colony-stimulating Factor-1 and Platelet-derived Growth Factor. J. Biol. Chem. 271(31); 18250-18354.
11) Ilaria, R. L., Jr, R. G. Hawley, and R. A. van Etten. 1999. Dominant Negative Mutants Implicate STAT5 in Myeloid Cell Proliferation and Neutrophil Differentiation. Blood. 93(12): 4154-4166.
12) Rosen, R. L., K. D. Winestock, G. Chen, X. Liu, L. Hennighausen, and D. S. Finbloom. 1996. Granulocyte-macrophage colony-stimulating factor preferentially activates the 94kD STAT5A and an 80kD STAT5A isoform in human peripheral blood monocytes. Blood. 88(4): 1207-1214.
13) Serreze, D. V., H. R. Gaskins, and E. H. Leiter. 1993. Defects in the differentiation and function of antigen presenting cells in the NOD/Lt Mice. J. Immunol. 150: 2534-43.
14) Morin, J., A. Chimenes, C. Boitard, R. Berthier, and S. Boudaly. 2003. Granulocyte-dendritic cell unbalance in the non-obese diabetic mice. Cell. Immunol. 223: 13-25.
15) Clare-Salzler, M. J. 1998. The immunopathogenic roles of antigen presenting cells in the NOD mouse. In: NOD mice and related strains: research applications in diabetes, AIDS, cancer and other diseases. editors: E. H. Leiter and M. A. Atkinson. Landes Bioscience Publishers, Austin, Tex. p. 101-120.
16) Feili-Hariri, M., and P. A. Morel. 2001. Phenotypic and Functional Characteristics of BM Derived DC from NOD and Non-Diabetes-Prone Strains. Clin Immunol. 98(1): 133-142.
17) Litherland, S. A., T. Xie, A. Hutson, D. S. Whittaker, D. Schatz, A. Haig, and M. Clare-Salzler. 1999. Aberrant Monocyte Prostaglandin Synthase 2 (PGS2) Expression Defines an Antigen Presenting Cell Defect and is a Novel Cellular Marker for Insulin Dependent Diabetes Mellitus (IDDM). J Clin Invest 104: 515-523.
18) Litherland, S. A., J.-X. She, D. Schatz, K. Fuller, A. D. Hutson, Y. Li, K. M. Grebe, D. S. Whittaker, K. Bahjat, D. Hopkins, Q. Fang, C. Wasserfall, R. Cook, M. A. Dennis, S. Crockett, J. Sleasman, J. Kocher, A. Muir, J. Silverstein, M. Atkinson, and M. J. Clare-Salzler. 2003. Aberrant Monocyte Prostaglandin Synthase 2 (PGS2) in Type 1 Diabetes Before & After Disease Onset. Pediatric Diabetes. 4: 10-18.
19) Litherland, S. A., T. X. Xie, Y. Li, K. M. Grebe, S. Reddy, L. L. Moldawer, H. Herschman, and M. J. Clare-Salzler. 2004. IL10 Resistant PGS2 Expression in At-Risk/Type 1 Diabetic Human Monocytes. J. Autoimmunity. 22(3):227-233.
20) Litherland, S. A., T. X. Xie, K. M. Grebe, A. Davoodi-Semiromi, J. Elf, N. S. Belkin, L. L. Moldawer, and M. J. Clare-Salzler. 2005. Signal Transduction Activator of Transcription 5 (STAT5) Proteins are Dysfunctional in Autoimmune Monocytes & Macrophages. J. Autoimmunity. 24: 297-310.
21) Abou-Raya, A. and S. Abou-Raya. 2006. Inflammation: a pivotal link between autoimmune diseases and atherosclerosis. Autoimmun. Rev. 5(5): 331.
22) Bouma, G., J. M. C. Coppens, W.-K. Lam-Tse, W. Luini, K. Sintnicalaas, W. H. Levering, S. Sozzani, H. A. Drexhage, and M. A. Versnel. 2005. An increased MRP8/14 expression and adhesion, but a decreased migration towards proinflammatory chemokines of type 1 diabetes monocytes. Clin & Exp Immunology. 141(3): 509-517.
23) Litherland, S. A., K. M. Grebe, N. S. Belkin, E. Paek, J. Elf, M. Atkinson, L. Morel, M. J. Clare-Salzler, and M. J. McDuffie. 2005. Nonobese diabetic mouse congenic analysis reveals chromosome 11 locus contributing to diabetes susceptibility, macrophage STAT5 dysfunction, and GM-CSF overproduction. J. Immunology. 175(7): 4561-4565.
24) Ito, K., P. J. Barnes, and I. M. Adcock. 2000. Glucocortoid Receptor Recruitment of Histone Deacetylase 2 Inhibits Interleukin-1b-Induced Histone H4 Acetylation on Lysines 8 and 12. Mol. Cell. Bio. 20(18): 6891-6903.
25) Chen, X., J. Wang, D. Woltring, S. Gerondakis, and M. F. Shannon. 2005. Histone Dynamics on the Interleukin-2 Gene in Response to T-Cell Activation. Mol Cell Biol. 25(8): 3209-3219.
26) Jacobsen, F. W., O. P. Veiby, and S. E. W. Jacobsen. 1994. IL-7 Stimulates CSF-Induced Proliferation of Murine Bone Marrow Macrophages and Mac-1+ Myeloid Progenitors In Vitro. J. Immunology. 153: 270-276.
27) Smale, S. T. 2003. The establishment and maintenance of lymphocyte identity through gene silencing. Nature Immunol. 4(7): 607-615.
28) Nakajima, H., P. K. Brindle, M. Handa, and J. N. Ihle. 2001. Functional interaction of STAT5 and nuclear receptor co-repressor SMRT: implications in negative regulation of STAT5-dependent transcription. 2001. EMBO J. 20(23)L 6836-6844.
29) Nelson, J. D., O. Denisenko, P. Soya, and K. Bomsztyk. 2006. Fast chromatin immunoprecipitation assay. Nucl. Acids Res. 34(1): e2-e7.
30) Schindler, C., Darnell, J. E., Jr. 1995. Transcriptional responses to polypeptide ligands: the JAK-STAT pathway. Annu. Rev. Biochem. 64: 621-65 1. Ehret, G. B., Reichenbach, R., Schindler, U., Horvath, C. M., Fritz, S., Nabholz, M., Buchner, P. 2001. DNA Binding Specificity of Different STAT Proteins. J. Biol. Chem. 276(9): 6675-6688.
31) Ehret, G. B., Reichenbach, R., Schindler, U., Horvath, C. M., Fritz, S., Nabholz, M., Buchner, P. 2001. DNA Binding Specificity of Different STAT Proteins. J. Biol. Chem. 276(9): 6675-6688.
32) Soldaini, E., John, S., Moro, S., Bollenbacher, J., Schindler, U., Leonard, W. J. 2000. DNA Binding Site Selection of Dimeric and Tetrameric STAT5Proteins Reveals a Large Repertoire of Divergent Tetrameric STAT5á Binding Sites. Mol. Cell. Biol. 20(1): 389-401.

We claim:

1. A method for identifying an individual at risk for developing an autoimmune disease wherein that method comprises determining whether the individual has dysfunctional STAT5 activity.

2. The method, according to claim 1, which further comprises evaluating whether the individual overproduces GM-CSF and/or displays aberrant PGS/COX2 expression/activity.

3. The method, according to claim 1, used to identify an individual at risk for developing type 1 diabetes.

4. The method, according to claim 1, wherein the individual is a human.

5. The method, according to claim 3, wherein, if the individual is identified as being at risk for developing type 1 diabetes, therapy is administered to the individual.

6. The method, according to claim 5, wherein the therapy comprises nutritional intervention.

7. The method, according to claim 1, comprising a genotype evaluation.

8. The method, according to claim 1, wherein said method comprises the use of flow cytometry.

9. The method, according to claim 2, which comprises the concurrent evaluation of STAT5, GM-CSF, and PGS/COX2.

10. A method for monitoring the effectiveness of a treatment for an autoimmune disorder in an individual wherein said method comprises monitoring the individuals' STAT5 activity.
